(19) United States
(12) Patent Application Publication
(10) Pub. No.: US 2011/0236349 A1
(43) Pub. Date: Sep. 29, 2011

(54) Use of Epidermal Growth Factor Inhibitors in the Treatment of Viral Infection

(76) Inventors: Jonathan L. Koff, San Francisco, CA (US); Jay A. Nadel, San Francisco, CA (US)

(21) Appl. No.: 13/058,999

(22) PCT Filed: Dec. 16, 2009
(86) PCT No.: PCT/US2009/068277

§ 371 Date: May 16, 2011

Related U.S. Application Data
(60) Provisional application No. 61/139,363, filed on Dec. 19, 2008.

(51) Int. Cl. A61K 38/21; A61K 31/517; A61K 31/4035; A61K 31/277; A61K 31/519; A61K 39/395; A61K 31/7088; A61P 31/12; A61P 31/22; A61P 31/16; A61P 31/14

(52) U.S. Cl. 424/85.5; 514/266.1; 514/417; 514/525; 514/265.1; 424/172.1; 514/44 R; 424/85.4; 424/85.7; 424/85.6

(57) ABSTRACT

The present invention provides methods of treating a viral infection in an individual. The methods generally involve administering to an individual an effective amount of an epidermal growth factor receptor (EGF-R) inhibitor.

### CROSS-REFERENCE

This application claims the benefit of U.S. Provisional Patent Application No. 61/139,363, filed Dec. 19, 2008, which application is incorporated herein by reference in its entirety.

### BACKGROUND

Viral infections cause considerable discomfort, disease and death. Viral infections target various human organs and systems such as the lungs and the gastrointestinal tract. Certain viruses, such as measles mumps, and chickenpox, are highly contagious and cause acute discomfort. Some viral infections lead to death. For example, influenza virus is responsible for the 1918 pandemic that has been cited as the most devastating epidemic in recorded world history. Influenza causes millions of infections worldwide each year and is responsible for up to 20,000 deaths per year in the United States. In addition, recent pandemics of avian influenza and Severe Acute Respiratory Syndrome (SARS), caused by a coronavirus, caused fatalities. Human parainfluenza virus (PIV) types 1, 2, and 3 and respiratory syncytial virus (RSV) types A and B are the major viral pathogens responsible for severe respiratory tract infections in infants and young children. It is estimated that, in the United States alone, approximately 1.6 million infants under one year of age will have a clinically significant RSV infection each year, and an additional 1.4 million infants will be infected with PIV-3. Approximately 4000 infants less than one year of age in the United States die each year from complications arising from severe respiratory tract disease caused by infection with RSV and PIV-3. Viral infections cause acute respiratory distress syndrome (ARDS) and acute lung injury, and cause exacerbations of chronic diseases such as asthma, chronic obstructive pulmonary disease (COPD), cystic fibrosis, and bronchiectasis. For example, rhinovirus is the most common cause of asthma exacerbations. Viral infections can also lead to chronic diseases. For example, human papilloma virus infection can lead to cervical cancer, and human immunodeficiency virus (HIV) is the cause of Acquired Immunodeficiency Syndrome (AIDS).

There is a need in the art for methods of treating viral infections.

### LITERATURE

WO 2005/048928; WO 2006/083458; Liu et al. (2008) J. Biol. Chem. 283:9977-9985; Tyner et al. J. Clin. Invest. (2006)116(2):309; Monick et al. J. Biol. Chem., (2005) 280:2147; Zhu et al. (2009) Am. J. Respir. Cell Mol. Biol. 40:610; U.S. Patent Publication No. 2007/0134763.

### SUMMARY OF THE INVENTION

The present disclosure provides methods of treating a viral infection in an individual. The methods generally involve administering to an individual an effective amount of an epidermal growth factor receptor inhibitor.

### BRIEF DESCRIPTION OF THE DRAWINGS

FIGS. 1A and 1B depict the effect of a selective epidermal growth factor receptor (EGF-R) inhibitor AG1478 (FIG. 1A); and a neutralizing antibody specific for EGF-R (FIG. 1B) on Rhinovirus 16 infection in airway epithelial cells.

FIG. 2 depicts flow cytometry data on the effect of a selective EGF-R inhibitor, AG1478, on Rhinovirus 16 infection in HeLa cells.

FIG. 3 depicts the effect of a selective EGF-R inhibitor, AG1478, on influenza virus infection of airway epithelial cells.

FIGS. 4A and 4B depict the effect of the selective EGF-R inhibitor AG1478, at 1 μM (FIG. 4A) or 10 μM (FIG. 4B), on respiratory syncytial virus (RSV) infection of epithelial cells.

FIG. 5 depicts the effect of the EGF-R-selective inhibitor Gefitinib on RSV infection of epithelial cells.

FIG. 6 provides an amino acid sequence of an EGF-R.

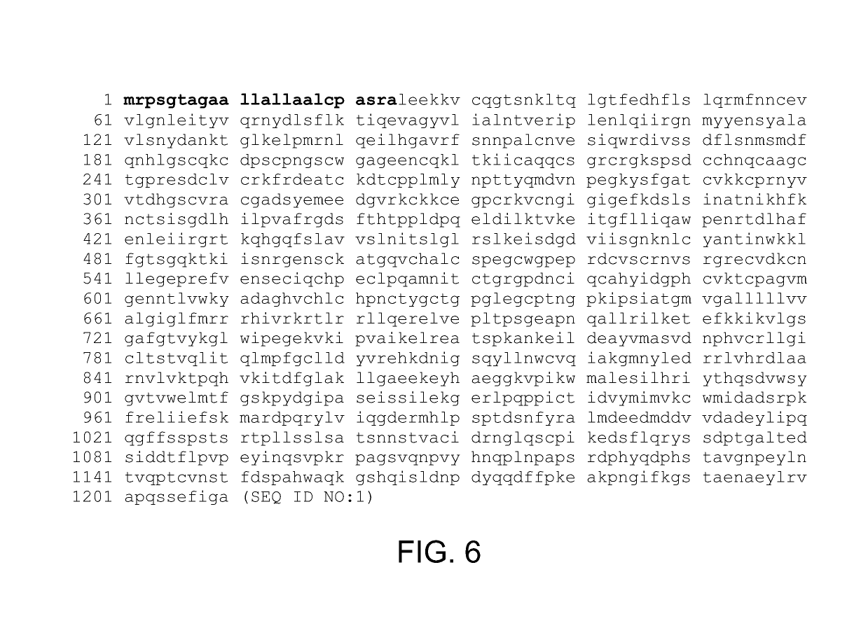

### DEFINITIONS

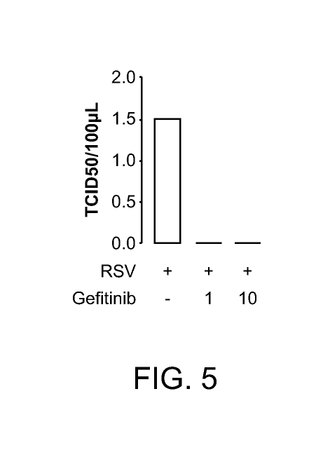

By “epidermal growth factor receptor” or “EGF-R” is meant a protein a portion thereof capable of binding epidermal growth factor (EGF) protein or a portion thereof. Exemplary is the human epidermal growth factor receptor (see Ullrich et al. (1984) Nature 309:418-425; Genbank accession number NM_{—}005228). The binding of EGF to EGF-R activates the EGF-R (e.g. resulting in autophosphorylation of EGF-R and activation of intracellular signaling). One of skill in the art will appreciate that other ligands, in addition to EGF, may bind to EGF-R and activate the EGF-R. Examples of such ligands include, but are not limited to, transforming growth factor-alpha (TGF-α), betacellulin, amphiregulin, heparin-binding EGF (HB-EGF) and neuregulin (also known as heregulin) (Strawn and Shawver (1998) Exp.-Opin. Invest. Drugs 7(4)553-573, and “The Protein Kinase Facts Book: Protein Tyrosine Kinases” (1995) Hardie, et al. (eds.), Academic Press, NY, N.Y.). A non-limiting example of an amino acid sequence of an EGF-R is depicted in FIG. 5 (GenBank NP_{—}005219; SEQ ID NO:5).

The terms “treatment,” “treating,” “treat,” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject who may be predisposed to the disease or symptom but has not yet been diagnosed as having it; (b) inhibiting the disease symptom, i.e., arresting its development; (c) relieving the disease symptom, i.e., causing regression of the disease or symptom; (d) limiting spread of a virus from one cell to another within an individual, e.g., limiting spread of a virus from an infected epithelial cell to other, uninfected, epithelial cells within an individual; (e) limiting replication of a virus within an individual; (f) limiting entry of a virus into a cell in an individual; and (g) reducing the number of viruses in an individual or in a target tissue or target biological sample in an individual.

The terms “subject,” “individual,” “host,” and “patient” are used interchangeably herein to refer to a mammal, including, but not limited to, murines (rats, mice), felines, non-human primates (e.g., simians), humans, canines, ungulates, etc. In some embodiments, an “individual” is a human, and can also be referred to as a “patient.”

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of compounds for use in a subject method calculated in an amount sufficient to produce the desired effect in association with a pharmaceutically acceptable diluent, carrier or vehicle. The specifications for the active agents for use in a subject method depend on the particular compound and the effect to be achieved, and the pharmacodynamics associated with each compound in the host.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectable system); and a single subcutaneous injection followed by installation of a continuous delivery system.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and generally free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by an oral route of administration. In some embodiments the composition is suitable for administration by an inhalation route of administration. In some embodiments the composition is suitable for administration by a transdermal route, e.g., using a penetration enhancer. In other embodiments, the pharmaceutical compositions are suitable for administration by a route other than transdermal administration.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “biological sample” encompasses a variety of sample types obtained from an individual. The definition encompasses blood, serum, plasma, and other liquid samples of biological origin; solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as epithelial cells. The term “biological, sample” encompasses a clinical sample, and also includes cells in culture, cell supernatants, organs, tissue samples, lung biopsy samples, lung epithelial cells, gastrointestinal epithelial cells, gastrointestinal tract tissue samples, bronchoalveolar lavage (BAL) fluid samples, nasal lavage fluid samples, blood, plasma, serum, cerebrospinal fluid, fecal samples, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a virus” includes a plurality of such a virus and reference to “the EGF-R inhibitor” includes reference to one or more EGF-R inhibitors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of treating a viral infection in an individual; and methods of treating acute exacerbations of chronic lung diseases, where the acute exacerbation is caused by a virus infection. The methods generally involve administering to an individual an effective amount of an epidermal growth factor receptor (EGF-R) inhibitor.

Current therapies for treating viral infections often treat symptoms, rather than the underlying infection. The present disclosure relates to treating a viral infection itself, involving administering an effective amount of an EGF-R inhibitor (also referred to herein as an “EGF-R antagonist”). Without being bound by theory, an EGF-R inhibitor can treat a viral infection in a virus receptor-independent manner, e.g., by blocking or inhibiting internalization of the virus into a cell, such as an epithelial cell, so as to inhibit infection of the cell by the virus and/or to inhibit replication of the virus in an individual. While a subject method of treating a viral infection can reduce a disease symptom of the viral infection, a subject method does not merely treat symptoms; instead, the viral infection is treated directly, e.g., by reducing internalization of a virus into a cell, thereby reducing, e.g., spread of the virus from an infected cell to an uninfected cell, viral replication, multiplication of the virus in a cell, etc.

### Methods of Treating a Viral Infection

The present disclosure provides methods of treating a viral infection in an individual. The methods generally involve administering to an individual in need thereof an effective amount of an epidermal growth factor receptor (EGF-R) inhibitor. The present disclosure further provides methods of treating virus-induced acute exacerbation of a chronic lung disease, the methods generally involving administering to an individual in need thereof (e.g., an individual having a chronic lung disease) an effective amount of an EGF-R inhibitor.

Administration of an effective amount of an EGF-R inhibitor to an individual having a virus infection results in one or more of: 1) a reduction in viral load; 2) a reduction in viral load in a target biological sample; 3) a reduction in the spread of a virus from one epithelial cell to another cell in an individual; 4) a reduction in viral entry into (e.g., reduction of internalization of a virus into) an epithelial cell; 5) a reduction in time to seroconversion (virus undetectable in patient serum); 6) an increase in the rate of sustained viral response to therapy; 7) a reduction of morbidity or mortality in clinical outcomes; and 8) an improvement in an indicator of disease response (e.g., a reduction in one or more symptoms of a viral infection, such as fever, etc.).

In some embodiments, an “effective amount” of an EGF-R inhibitor is an amount that, when administered in one or more doses to an individual having a virus infection, is effective to reduce the number of genome copies of the virus in the individual, e.g., in a target biological sample in the individual. For example, in some embodiments, an “effective amount” of an EGF-R inhibitor is an amount that, when administered in one or more doses to an individual having a virus infection, is effective to reduce the number of genome copies of the virus in the individual by at least about 20%, at least about 25%, at least about 30%, at least about 40%, at least about 50%, at least about 60%, at least about 70%, at least about 80%, at least about 90%, or more than 90%, compared to the number of genome copies in the individual in the absence of treatment with the inhibitor.

For example, in some embodiments, an “effective amount” of an EGF-R inhibitor is an amount that, when administered in one or more doses to an individual having a virus infection, is effective to reduce the number of genome copies of the virus present in a biological sample obtained from the individual by at least about 20%, at least about 25%, at least about 30%, at least about 40%, at least about 50%, at least about 60%, at least about 70%, at least about 80%, at least about 90%, or more than 90%, compared to the number of genome copies in the biological sample in the absence of treatment with the inhibitor. Biological samples include, e.g., lung samples (e.g., where the virus is a respiratory virus), where exemplary lung samples include, e.g., BAL fluid, epithelial cells obtained from the lung, lung biopsy tissue, etc.; nasal samples (e.g., where the virus is a respiratory virus), where exemplary nasal samples include nasal swabs, nasal lavage samples, cells obtained from a nasal passage, etc.; oropharynx samples (e.g., where the virus is a respiratory virus), where exemplary oropharynx samples include oral swabs, oral lavage samples, and cells (e.g., epithelial cells) obtained from the mouth, e.g., by brush or biopsy, etc.; a gastrointestinal tract sample (e.g., where the virus is a gastrointestinal virus), where exemplary gastrointestinal tract samples include a stool sample (e.g., fecal matter), biopsy tissue obtained from the gastrointestinal tract, cells obtained from the gastrointestinal tract, etc.; mucosal tissue samples (e.g., where the virus infects a mucosal tissue) include vaginal samples (e.g., cells obtained from the vagina), gastrointestinal tract samples, lower gastrointestinal tract samples, lung samples, oropharynx samples, etc.

For example, in some embodiments, an “effective amount” of an EGF-R inhibitor is an amount that, when administered in one or more doses to an individual having a virus infection, is effective to reduce the number of genome copies of the virus in a mucosal tissue (e.g., a gastrointestinal tract tissue; a lung tissue) in the individual by at least about 20%, at least about 25%, at least about 30%, at least about 40%, at least about 50%, at least about 60%, at least about 70%, at least about 80%, at least about 90%, or more than 90%, compared to the number of genome copies in the mucosal tissue in the individual in the absence of treatment with the inhibitor.

As another example, in some embodiments, an “effective amount” of an EGF-R inhibitor is an amount that, when administered in one or more doses to an individual having a respiratory virus infection, is effective to reduce the number of genome copies of the respiratory virus in a lung biological sample or a nasal biological sample in the individual by at least about 20%, at least about 25%, at least about 30%, at least about 40%, at least about 50%, at least about 60%, at least about 70%, at least about 80%, at least about 90%, or more than 90%, compared to the number of genome copies in the lung biological sample or nasal biological sample in the individual in the absence of treatment with the inhibitor.

As another example, in some embodiments, an “effective amount” of an EGF-R inhibitor is an amount that, when administered in one or more doses to an individual having a gastrointestinal tract virus infection, is effective to reduce the number of genome copies of the gastrointestinal tract virus in a gastrointestinal tract biological sample in the individual by at least about 20%, at least about 25%, at least about 30%, at least about 40%, at least about 50%, at least about 60%, at least about 70%, at least about 80%, at least about 90%, or more than 90%, compared to the number of genome copies in the gastrointestinal tract biological sample in the individual in the absence of treatment with the inhibitor.

In some embodiments, an “effective amount” of an EGF-R inhibitor is an amount that, when administered in one or more doses to an individual having a virus infection, is effective to reduce the number of genome copies of the virus in the individual to from about 1000 genome copies/mL serum to about 5000 genome copies/mL serum, to from about 500 genome copies/mL serum to about 1000 genome copies/mL serum, to from about 100 genome copies/mL serum to about 500 genome copies/mL serum, or to less than 100 genome copies/mL serum. In some embodiments, an “effective amount” of an EGF-R inhibitor is an amount that, when administered in one or more doses to an individual having a virus infection, is effective to achieve a 1.5-log, a 2-log, a 2.5-log, a 3-log, a 3.5-log, a 4-log, a 4.5-log, or a 5-log reduction in viral titer in the serum of the individual.

In some embodiments, an effective amount of an EGF-R inhibitor is an amount that reduces or inhibits spread of a virus from an infected epithelial cell to uninfected epithelial cells in a virus-infected individual. For example, in some embodiments, an effective amount of an EGF-R inhibitor is an amount that reduces or inhibits spread of a virus from an infected epithelial cell to uninfected epithelial cells in a virus-infected individual by at least about 20%, at least about 25%, at least about 30%, at least about 40%, at least about 50%, at least about 60%, at least about 70%, at least about 80%, at least about 90%, or more than 90%, compared to the spread of the virus in the absence of treatment with the inhibitor. For example, in some embodiments, an effective amount of an EGF-R inhibitor is an amount that prevents an uninfected epithelial cell in an individual from becoming infected with virus present in the individual.

In some embodiments, an effective amount of an EGF-R inhibitor is an amount that reduces replication of a virus in a virus-infected individual. For example, in some embodiments, an effective amount of an EGF-R inhibitor is an amount that reduces replication of a virus in a virus-infected individual by at least about 20%, at least about 25%, at least about 30%, at least about 40%, at least about 50%, at least about 60%, at least about 70%, at least about 80%, at least about 90%, or more than 90%, compared to the amount of replication in the absence of treatment with the inhibitor. An effect on viral replication can be determined by measuring viral load in a biological sample obtained from an individual.

In some embodiments, an effective amount of an EGF-R inhibitor is an amount that reduces the severity of disease (e.g., disease symptoms) experienced by an individual infected with a virus. For example, in some embodiments, an effective amount of an EGF-R inhibitor is an amount that is effective to reduce the severity of a disease caused by a virus in a virus-infected individual, e.g., is effective to reduce the severity of an adverse symptom of a viral infection by at least about 20%, at least about 25%, at least about 30%, at least about 40%, at least about 50%, at least about 60%, at least about 70%, at least about 80%, at least about 90%, or more than 90%, compared to the severity of the disease (e.g., adverse disease symptom) experienced by the individual not treated with the inhibitor. Adverse disease symptoms include, e.g., fever, cough, difficulty breathing, vomiting, diarrhea, muscle aches, excess lung fluid, headache, and the like.

In some embodiments, an effective amount of an EGF-R inhibitor is an amount that reduces the risk that a person who has been exposed to a virus, but who has not yet exhibited symptoms of infection by the virus, will develop disease symptoms resulting from infection by the virus.

In some embodiments, an effective amount of an EGF-R inhibitor is an amount that reduces the time to viral clearance by at least about 10%, at least about 20%, at least about 25%, at least about 30%, at least about 35%, at least about 40%, at least about 50%, at least about 60%, at least about 70%, at least about 80%, at least about 90%, or more, compared to the time to viral clearance in the absence of treatment with the EGF-R inhibitor.

In some embodiments, an effective amount of an EGF-R inhibitor is an amount that reduces morbidity or mortality due to a virus infection by at least about 10%, at least about 20%, at least about 25%, at least about 30%, at least about 35%, at least about 40%, at least about 50%, at least about 60%, at least about 70%, at least about 80%, at least about 90%, or more, compared to the morbidity or mortality in the absence of treatment with the EGF-R inhibitor.

Whether a subject treatment method is effective in reducing viral load, reducing time to viral clearance, or reducing morbidity or mortality due to a virus infection is readily determined by those skilled in the art. Viral load is readily measured by measuring the titer or level of virus in serum or other target biological sample(s). The number of viruses in the serum or other target biological sample(s) can be determined using any known assay, including, e.g., a quantitative polymerase chain reaction (qPCR) assay using oligonucleotide primers specific for the virus being assayed, a viral plaque assay, tissue culture infective dose 50 (TCID₅₀) assay, etc. Whether morbidity is reduced can be determined by measuring any symptom associated with a virus infection, including, e.g., fever, respiratory symptoms (e.g., cough, ease or difficulty of breathing, and the like), gastrointestinal symptoms, etc. The TCID₅₀ is the median tissue culture infective dose; e.g., that amount of a pathogenic agent that will produce pathological change in 50% of cell cultures inoculated; and can be expressed as TCID₅₀/ml (see, e.g., Reed and Muench (1938) Am. J. Hyg. 27:493).

In some embodiments, the present disclosure provides methods of reducing viral load, and/or reducing the time to viral clearance, and/or reducing morbidity or mortality in an individual who has not been infected with a virus, and who has been exposed to a virus. In some of these embodiments, the methods involve administering an effective amount of an EGF-R inhibitor within 48 hours of exposure to the virus. In other embodiments, the methods involve administering an EGF-R inhibitor more than 48 hours after exposure to the virus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the virus.

A therapeutic regimen comprises administering to an individual in need thereof a therapeutically effective amount of an EGF-R inhibitor. In some embodiments, multiple doses of an EGF-R inhibitor are administered. The frequency of administration of an EGF-R inhibitor can vary, depending on any of a variety of factors, e.g., severity of the symptoms, etc. For example, in some embodiments, an EGF-R inhibitor is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an EGF-R inhibitor, e.g., the period of time over which an EGF-R inhibitor is administered, can vary, depending on any of a variety of factors, e.g., severity of symptoms, patient response, etc. For example, an EGF-R inhibitor can be administered over a period of time ranging from about one day to about 2 days, from about 2 days to about 4 days, from about 4 days to about one week, from about two weeks to about four weeks, from about one month to about two months, from about two months to about four months, or longer than four months.

### EGF-R Inhibitors

EGF-R inhibitors that are suitable for use in a subject method include any agent capable of directly or indirectly inhibiting activation of EGF-R. EGF-R can be activated through ligand-dependent and ligand-independent mechanisms, resulting in either trans-phosphorylation or autophosphorylation, respectively. EGF-R antagonists of interest can inhibit either or both of these mechanisms. In other embodiments, a suitable EGF-R antagonist reduces activation of EGF-R.

Suitable EGF-R inhibitors include a small molecule EGF-R antagonist; an antibody that specifically binds EGF-R and reduces activation of EGF-R, e.g., by blocking binding of a ligand to EGF-R; an antibody that specifically binds an EGF-R agonist and blocks binding of the EGF-R agonist to the EGF-R; and an inhibitory nucleic acid that specifically reduces production of EGF-R.

### Small Molecule Inhibitors

Small molecule EGF-R inhibitors include, e.g., compounds that are less than about 25 kDa, e.g., compounds that are from about 50 daltons to about 25 kDa, e.g., from about 50 daltons to about 100 daltons, from about 100 daltons to about 500 daltons, from about 500 daltons to about 1 kilodaltons (kDa), from about 1 kDa to about 5 kDa, from about 5 kDa to about 10 kDa, or from about 10 kDa to about 25 kDa. Small molecule inhibitors can have a molecular weight in a range of from about 50 daltons to about 3000 daltons, e.g., from about 50 daltons to about 75 daltons, from about 75 daltons to about 100 daltons, from about 100 daltons to about 250 daltons, from about 250 daltons to about 500 daltons, from about 500 daltons to about 750 daltons, from about 750 daltons to about 1000 daltons, from about 1000 daltons to about 1250 daltons, from about 1250 daltons to about 1500 daltons, from about 1500 daltons to about 2000 daltons, from about 2000 daltons to about 2500 daltons, or from about 2500 daltons to about 3000 daltons. In some embodiments, a small molecule EGF-R inhibitor is not a peptide.

A small molecule tyrosine kinase inhibitor that is an EGF-R antagonist can have an IC₅₀ (half maximal effective inhibitory concentration) from about 1 pM to about 1 mM, e.g., from about 1 pM to about 10 pM, from about 10 pM to about 25 pM, from about 25 pM to about 50 pM, from about 50 pM to about 100 pM, from about 100 pM to about 250 pM, from about 250 pM to about 500 pM, from about 500 pM to about 750 pM, from about 750 pM to about 1 nM, from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM, from about 250 μM to about 500 μM, or from about 500 μM to about 1 mM. In some embodiments, a suitable EGF-R antagonist is a tyrosine kinase inhibitor that has an IC₅₀ of from about 1 μM to about 1 nM. In some embodiments, a suitable EGF-R antagonist is a tyrosine kinase inhibitor that has an IC₅₀ of from about 1 nM to about 1 μM. In some embodiments, a suitable EGF-R antagonist is a tyrosine kinase inhibitor that has an IC₅₀ of from about 1 μM to about 500 μM. In some embodiments, a suitable EGF-R antagonist is a tyrosine kinase inhibitor that has an IC₅₀ of from about 500 μM to about 1 mM.

In some embodiments, a suitable small molecule EGF-R inhibitor is a tyrosine kinase inhibitor that is selective for EGF-R. The term “selective” in the context of a “tyrosine kinase inhibitor that is selective for EGF-R” is a term well understood by those skilled in the art. For example, a tyrosine kinase inhibitor that is selective for EGF-R inhibits EGF-R to a greater degree than other cell surface receptors having tyrosine kinase activity, e.g., a tyrosine kinase inhibitor that is selective for EGF-R inhibits the tyrosine kinase activity a cell surface receptor having tyrosine kinase activity (other than an EGF-R), if at all, by less than about 20%, less than about 15%, less than about 10%, or less than about 5%, at a concentration that would cause at least a 50% inhibition of tyrosine kinase activity of an EGF-R. Receptor tyrosine kinases (other than EGF-R) include, e.g., ErbB-2, ErbB-3, ErbB-4; a member of an insulin receptor tyrosine kinase (RTK) family; a member of a platelet derived growth factor (PDGF) RTK family; a member of a fibroblast growth factor RTK family; a member of a vascular endothelial growth factor RTK family; a TrkA, TrkB, or TrkC RTK; and the like.

In some embodiments, a tyrosine kinase inhibitor that is selective for EGF-R inhibits EGF-R to a greater degree than a non-receptor tyrosine kinase, where non-receptor tyrosine kinases include, e.g., a member of SRC family tyrosine kinase (TK), where SRC family TK members include, e.g., B lymphoid tyrosine kinase (BLK), breast tumor kinase/protein tyrosine kinase 6 (BrK/PTK6), Gardner-Rasheed feline sarcoma viral oncogene homolog (FGR), Fyn oncogene related to Src, FGR, Yes (Fyn), hemopoietic cell kinase (HCK), LcK, v-Yes-1, Yamaguchi sarcoma viral-related oncogene homolog (Lyn), Src, Src-related kinase lacking C-terminal regulatory tyrosine and N-terminal myristoylation sites (SRMS), Yes, and Yes-related kinase (YRK)); a member of a JAK family TK (e.g., JaK1, JaK2, TyK2, etc.); a member of an ABL family TK; a member of a FAK family TK (e.g., FAK, PYK2/CAKβ, etc.); a member of an FPS family TK; a member of a CSK family TK; a member of a SYK family TK; and a member of a BTK family TK. In some embodiments, a tyrosine kinase inhibitor that is selective for EGF-R inhibits a non-receptor TK, if at all, by less than about 20%, less than about 15%, less than about 10%, or less than about 5%, at a concentration that would cause at least a 50% inhibition of tyrosine kinase activity of an EGF-R.

In some embodiments, a suitable EGF-R tyrosine kinase inhibitor inhibits the activity of one, two, three, or four receptor tyrosine kinases in addition to EGF-R tyrosine kinase. In some embodiments, a suitable EGF-R tyrosine kinase inhibitor inhibits the activity of one or two non-receptor tyrosine kinases in addition to EGF-R tyrosine kinase.

Suitable EGF-R inhibitors include those described in WO99/09016 (American Cyanamid); WO98/43960 (American Cyanamid); WO97/38983 (Warner Lambert); WO99/06378 (Warner Lambert); WO99/06396 (Warner Lambert); WO96/30347 (Pfizer, Inc.); WO96/33978 (Zeneca); WO96/33977 (Zeneca); and WO96/33980); U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, 5,747,498, WO98/14451, WO98/50038, WO99/09016, and WO99/24037.

Suitable EGF-R inhibitors include quinazolines and quinazoline derivatives. Exemplary quinazolines are PD 153035, 4-(3-chloroanilino) quinazoline, and CP-358,774. The structures of a number of quinazoline EGF-R inhibitors are known in the art. See, e.g., Fry et al. (1994) Science 265:1093 for a description of PD 153035; and Moyer et al. (1997) Cancer Res. 57:4838 for a description of CP-358,774. PD153035 is 4-[(3-bromophenyl)amino]-6,7-dimethoxyquinazoline.

Suitable EGF-R inhibitors include quinazoline derivatives. An exemplary quinazoline derivative is ZD1839 (Gefitinib; Iressa; N-(3-chloro-4-fluoro-phenyl)-7-methoxy-6-(3-morpholin-4-ylpropoxy)quinazolin-4-amine). The structures of various quinazoline derivatives that are EGF-R inhibitors are known. ZD1839 is known in the art. See, e.g., U.S. Pat. No. 5,770,599, and Strawn and Shawver (April 1998) Exp. Opinion Invest. Drugs 7:553, for the structure of ZD1839. Another example of a suitable quinazoline derivative is Tarceva™ (OSI-774; also referred to as CP-358774 or erlotinib), a 4-anilinoquinazoline derivative. CP-358774 is ([6,7-bis(2-methoxyethoxy)-4-quinazolin-4-yl]-(3-ethynylphenyl)amine. Salts of such compounds, e.g., hydrochloride salt (e.g., erlotinib HCl), and other salt forms (e.g., erlotinib mesylate) are also suitable for use. See, e.g., Morin (2000) Oncogene 19:6574 for the structure of CP-358774. ZD6474 is also suitable for use. Vandetanib (ZACTIMA™; ZD6474) is a dual VEGFR2 and EGF-R tyrosine kinase inhibitor. ZD6474 is 4-(4-bromo-2-fluoroanilino)-6-methoxy-7-(1-methylpiperidin-4-ylmethoxy) quinazoline.

Suitable EGF-R inhibitors include substituted diaminophthalimides. An exemplary substituted diaminophthalimide is 4,5-bis(4-fluoroanilino)phthalamide. See, e.g., Buchdunger et al. (1995) Clin. Cancer Res. 1:813.

Suitable EGF-R inhibitors include tyrphostins. See, e.g., Levitzki and Gazit (1995) Science 267:1782; and Ben-Bassat (1997) Cancer Res. 57:3741. In some embodiments, the tyrphostin is AG1478 (4-(3-chlorophenylamino)-6,7-dimethoxyquinazoline). An exemplary tyrphostin is AG1571 (SU 5271; Sugen); the structure of AG1571 is found in, e.g., Huang et al. (2003) J. Pharmacol. Exp. Ther. 304:753.

Suitable EGF-R inhibitors include pyrrolopyrimidines, including the 7H-pyrrolo[2,3] class of pyrimidines. Exemplary pyrrolopyrimidines include, e.g., 4-(phenylamino)-7H-pyrrolo[2,3-d]pyrimidine, CGP 59326, CGP 60261, and CGP 62706. See, e.g., Traxler et al. (1996) J. Med. Chem. 39:2285. See, e.g., Traxler et al. (1997) J. Pharm. Belg. 52:88 for the structures of CGP 59326, CGP 60261, and CGP 62706. Another suitable pyrrolopyrimidine is PKI-166 (CGP 75166), which is (R)-4-[4-[(1-phenylethyl)amino]-7H-pyrrolo[2,3-d]pyrimidin-6-yl]-phenol. See, e.g., Hoekstra et al. (2005) Clin. Cancer Res. 11:6908 for a description of CGP 75166. Another suitable pyrrolopyrimidine EGF-R tyrosine kinase inhibitor is (R)-6-(4-hydroxyphenyl)-4-[(1-phenylethyl)amino]-7H-pyrrolo[2,3-d]pyrimidine). Also suitable for use is AEE788. AEE788 inhibits phosphorylation of EGF-R, HER2, and VEGF-R2 tyrosine kinases. AEE788 is a member of the 7H-pyrrolo[2,3] class of pyrimidines. AEE788 is [6-[4-[(4-ethylpiperazin-1-yl)methyl]phenyl]-7H-pyrrolo[2,3-d]pyrimidin-4-yl]-((R)-1-phenylethyl)amine.

Suitable EGF-R inhibitors can be of any of a variety of chemical classes, including, e.g., a quinazoline, a pyridopyrimidine, a pyrimidopyrimidine, a pyrrolopyrimidine, a pyrazolopyrimidine, a diaminophthalimide, a bicyclic heterocyclic compound, and a tyrphostin. Thus, in some embodiments, an EGF-R inhibitor is selected from a quinazoline, a pyridopyrimidine, a pyrimidopyrimidine, a pyrrolopyrimidine, a pyrazolopyrimidine, a diaminophthalimide, a bicyclic heterocyclic compound, and a tyrphostin.

Suitable EGF-R inhibitors include bicyclic heterocyclic compounds such as those described in WO 2008/05584, WO 2008/095847, U.S. Patent Publication No. 2007/0185081, and U.S. Patent Publication No. 2007/0185091.

For example, compounds of the general formula:

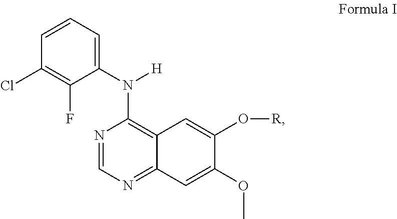

where R is as described in WO 2008/055854, are suitable for use. For example, as described in WO 2008/055854, R of Formula I can be cis-4-amino-cyclohexyl, trans-4-amino-cyclohexyl, cis-4-methylamino-cyclohexyl, trans-4-methylamino-cyclohexyl, cis-4-(methoxycarbonylamino)-cyclohexyl, trans-4 (methoxycarbonylamino)-cyclohexyl, cis-4-(N-methoxycarbonyl-N-methylamino)-cyclohexyl, trans-4-(N-methoxycarbonyl-N-methyl-amino)-cyclohexyl, cis-4-(ethyloxycarbonylamino)-cyclohexyl, trans-4-(ethyloxycarbonylamino)-cyclohexyl, cis-4-(N-ethyloxycarbonyl-N-methyl-amino)-cyclohexyl, trans-4-(N-ethyloxycarbonyl-N-methylamino)-cyclohexyl, cis-4-(tert-butoxycarbonylamino)-cyclohexyl, trans-4-(tert-butoxycarbonylamino)-cyclohexyl, cis-4-(tert-butoxylcarbonyl-N-methylamino)-cyclohexyl, trans-4-(tert-butoxycarbonyl-N-methylamino)-cyclohexyl, cis-4-(acetylamino)-cyclohexyl, trans-4-(acetylamino)-cyclohexyl, cis-4-(N-acetyl-N-methyl-amino)-cyclohexyl, trans-4-(N-acetyl-N-methyl-amino)-cyclohexyl, cis-4-(methoxyacetyl-amino)-cyclohexyl, trans-4-(methoxyacetyl-amino)-cyclohexyl, cis-4-(N-methoxyacetyl-N-methyl-amino)-cyclohexyl, trans-4-(N-methoxyacetyl-N-methyl-amino)-cyclohexyl, cis-4-(dimethylaminocarbonyl-amino)-cyclohexyl, trans-4-(dimethylaminocarbonylamino)-cyclohexyl, cis-4-(N-dimethylaminocarbonyl-N-methyl-amino)-cyclohexyl; trans-4-(N-dimethylaminocarbonyl-N-methyl-amino-cyclohexyl, cis-4-(morpholinocarbonyl-amino)-cyclohexyl, trans-4-(morpholinocarbonyl-amino)-cyclohexyl, cis-4-(N-morpholinocarbonyl-N-methyl-amino)-cyclohexyl, trans-4-(N-morpholinocarbonyl-N-methyl-amino-cyclohexyl, cis-4-(piperazin-1-ylcarbonylamino)-cyclohexyl, trans-4-(piperazin-1-ylcarbonylamino)-cyclohexyl, cis-4-(N-piperazin-1-ylcarbonyl-N-methylamino)-cyclohexyl, trans-4-(N-piperazin-1-ylcarbonyl-N-methylamino)-cyclohexyl, cis-4-[(4-methyl-piperazin-1-ylcarbonyl)-amino]-cyclohexyl, trans-4-[(4-methyl-piperazin-1-ylcarbonyl)-amino]-cyclohexyl, cis-4 [N-(4-methyl-piperazin-1-ylcarbonyl)-N-methyl-amino]-cyclohexyl, trans-4-[N-(4-methyl-piperazin-1-ylcarbonyl)-N-methyl-amino]-cyclohexyl, cis-(methansulfonylamino)-cyclohexyl, trans-4-(methansulfonylamino)-cyclohexyl, cis-4-(N-methansulfonyl-N-methyl-amino)-cyclohexyl, trans-4-(N-methansulfonyl-N-methyl-amino)-cyclohexyl, cis-4-phthalimido-cyclohexyl, and trans-4-phthalimido-cyclohexyl. For example, in some embodiments, a suitable EGF-R antagonist is a compound of the formula:

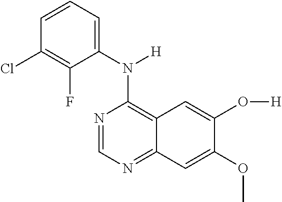

As another example, compounds of the general formula:

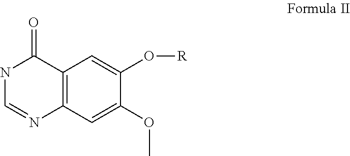

where R is as described in WO 2008/055854, are suitable for use. For example, as described in WO 2008/055854, R of Formula II can be as described for R of Formula I above.

As another example, compounds of the general formula:

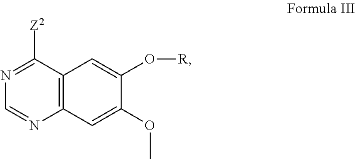

where Z² and R are as described in WO 2008/055854, are suitable for use. For example, as described in WO 2008/055854, R of Formula III can be as described for R of Formula I above.

As another example, compounds of the general formula:

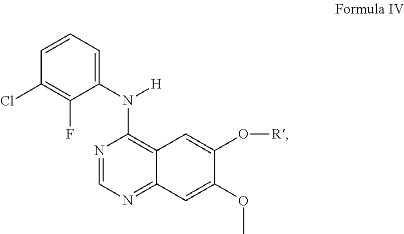

where R′ is as described in WO 2008/055854, are suitable for use. For example, as described in WO 2008/055854, R′ of Formula IV can be cis-4-amino-cyclohex-1-yl, trans-4-amino-cyclohex-1-yl, cis-4-(methylamino)-cyclohex-1-yl or trans-4-(methylamino)-cyclohex-1-yl.

As another example, compounds of the general formula:

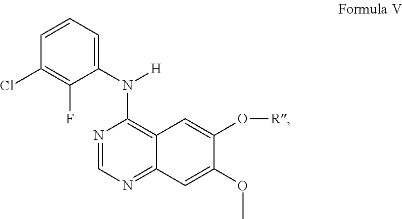

where R″ is as described in WO 2008/055854, are suitable for use. For example, as described in WO 2008/055854, R″ of Formula V can be cis-4-amino-cyclohex-1-yl or trans-4-amino-cyclohex-1-yl.

As another example, compounds of the general formula

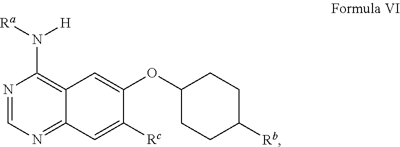

where R^{a}, R^{b}, and R^{c} are as described in WO 2008/095847, are suitable for use.

For example, a compound of Formula VI, as described in WO 2008/055854, where:

R^{a} can be a phenyl, 1-phenylethyl or indan-4-yl group, where the phenyl nucleus is substituted in each case by the groups R¹ to R³,
where R¹ and R², can be the same or different, and each is selected from a hydrogen, fluorine, chlorine, bromine, or iodine atom, a C₁₋₄-alkyl, hydroxy, a C₁₋₄-alkoxy-, a C₂₋₃-alkenyl, a C₂₋₃-alkynyl, an aryl, an aryloxy, an arylmethyl, an arylmethoxy, a heteroaryl, a heteroaryloxy, a heteroarylmethyl, a heteroarylmethoxy, a methyl or methoxy substituted by 1 to 3 fluorine atoms, cyano, nitro, or an amino, and
R³ is a hydrogen, fluorine, chlorine, or bromine atom, or a methyl or trifluoromethyl; and
R^{b} can be azetidin-1-yl, pyrrolidin-1-yl, piperidin-1-yl, homopiperidin-1-yl, morpholin-4-yl, homomorpholin-4-yl, piperazin-1-yl, 4-(C₁₋₄-alkyl-carbonyl)-piperazin-1-yl, 4-(C₁₋₄-alkyl-sulfonyl)-piperazin-1-yl, homopiperazin-1-yl, 4-(C₁₋₄-alkyl-carbonyl)-homopiperazin-1-yl or 4-(C₁₋₄-alkyl-sulfonyl)-homopiperazin-1-yl, which may be mono-, di- or tri-substituted by R⁴ in each case; where
the substitutents may be identical or different and where R⁴ is a fluorine, chlorine, bromine or iodine atom, a C₁₋₄-alkyl, C₂₋₄-alkenyl or C₂₋₄-alkynyl group, a methyl or methoxy group substituted by 1 to 3 fluorine atoms, an amino, C₁₋₄-alkylamino, di-(C₁₋₄-alkyl)amino, C₁₋₄-alkyl-carbonylamino, N—(C₁₋₄-alkyl)-C₁₋₄-alkyl-carbonylamino, C₁₋₄,-alkyl-sulfonylamino or N—(C₁₋₄-alkyl)-C₁₋₄-alkyl-sulfonylamino group, an amino-C₁₋₄-alkyl, C₁₋₄-alkylamino-C₁₋₄-alkyl, di-(C₁₋₄-alkyl)amino-C₁₋₄-alkyl, C₁₋₄-alkyl-carbonylamino-C₁₋₄-alkyl, N—(C₁₋₄-alkyl)-C₁₋₄-alkyl-carbonylamino-C₁₋₄-alkyl-, C₁₋₄-alkyl-sulfonylamino-C₁₋₄-alkyl or N—(C₁₋₄-alkyl)-C₁₋₄-alkyl-sulfonylamino-C₁₋₄-alkyl group, a hydroxy, C₁₋₄-alkyloxy or C₁₋₄-alkyl-carbonyloxy group, a hydroxy-C₁₋₄-alkyl, C₁₋₄-alkyloxy-C₁₋₄-alkyl or C₁₋₄-alkyl-carbonyloxy-C₁₋₄-alkyl group, a C₁₋₄-alkyl-carbonyl, cyano, C₁₋₄-alkyl-oxycarbonyl, carboxy, aminocarbonyl, C₁₋₄-alkyl-aminocarbonyl, di-(C₁₋₄-alkyl)amino-carbonyl, pyrrolidin-1-yl-carbonyl, piperidin-1-yl-carbonyl, piperazin-1-yl-carbonyl, 4-C₁₋₄-alkyl-piperazin-1-yl-carbonyl or morpholin-4-yl-carbonyl group, a C₁₋₄-alkylcarbonyl-C₁₋₄-alkyl, cyano-C₁₋₄-alkyl, C₁₋₄-alkyloxycarbonyl-C₁₋₄-alkyl, aminocarbonyl-C₁₋₄-alkyl, C₁₋₄-alkylaminocarbonyl-C₁₋₄-alkyl, di-(C₁₋₄-alkyl)aminocarbonyl-C₁₋₄-alkyl, pyrrolidin-1-yl-carbonyl-C₁₋₄-alkyl-, piperidin-1-yl-carbonyl-C₁₋₄-alkyl, piperazin-1-yl-carbonyl-C₁₋₄-alkyl, 4-C₁₋₄-alkyl-piperazin-1-yl-carbonyl-C₁₋₄-alkyl or morpholin-4-yl-carbonyl-C₁₋₄-alkyl group, a C₁₋₄-alkylsulfanyl, C₁₋₄-alkylsulfinyl, C₁₋₄-alkylsulfonyl, aminosulfonyl, C₁₋₄-alkyl-aminosulfonyl or di-(C₁₋₄-alkyl)amino-sulfonyl group, a C₁₋₄-alkylsulfanyl-C₁₋₄-alkyl, C₁₋₄-alkylsulfinyl-C₁₋₄-alkyl, C₁₋₄-alkylsulfonyl-C₁₋₄-alkyl, aminosulfonyl-C₁₋₄-alkyl, C₁₋₄-alkyl-aminosulfonyl-C₁₋₄-alkyl or di-(C₁₋₄-alkyl)amino-sulfonyl-C₁₋₄-alkyl group; and where the heterocycles mentioned above under R^{b} may additionally be substituted by an oxo group,
R^{c} can be a hydrogen atom, fluorine, chlorine, bromine or iodine atom;
a C₁₋₄-alkyl group or a C₁₋₄-alkyl group, which is substituted by an R⁵ group,
where R⁵ is a hydroxy, C₁₋₃-alkyloxy, C₃₋₆-cycloalkyloxy,a-, C₁₋₃-alkylamino, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza bicyclo[2.2.1]hept-5-yl, 3-oxa-8-aza-bicyclo[3.2.1]oct-8-yl, 8-oxa-3-aza-bicyclo[3.2.1]oct-3-yl, piperazin-1-yl-, 4-C₁₋₃-alkyl-piperazin-1-yl, homopiperazin-1-yl or C₁₋₃-alkyl-homopiperazin-1-yl group or a formylamino, C₁₋₄-alkylcarbonylamino, C₁₋₃-alkyloxy-C₁₋₃-alkyl-carbonylamino, C₁₋₄-alkyloxycarbonylamino, aminocarbonylamino, C₁₋₃-alkylaminocarbonylamino, di-(C₁₋₃-alkyl)aminocarbonylamino, pyrrolidin-1-ylcarbonylamino, piperidin-1-ylcarbonylamino, piperazin-1-ylcarbonylamino, 4-C₁₋₃-alkyl-piperazin-1-ylcarbonylamino, morpholin-4-ylcarbonylamino or a C₁₋₄-alkylsulfonylamino group;
a hydroxy, a C₁₋₄-alkyloxy, a methoxy or ethyloxy group substituted by 1 to 3 fluorine atoms, a C₂₋₄-alkyloxy group which is substituted by the group R⁵, where R⁵ is as hereinbefore defined, a C₃₋₇-cycloalkyloxy or C₃₋₄-cycloalkyl-C₁₋₄-alkyloxy group, a tetrahydrofuran-3-yloxy, tetrahydropyran-3-yloxy or tetrahydropyran-4-yloxy group, a tetrahydrofuranyl-C₁₋₄-alkyloxy or tetrahydropyranyl-C₁₋₄-alkyloxy group;
a C₁₋₄-alkoxy group which is substituted by a pyrrolidinyl, piperidinyl or homopiperidinyl group substituted in the 1 position by the R⁶ group, where
R⁶ is a hydrogen atom or a C₁₋₃-alkyl group;
or a C₁₋₄-alkoxy group, which is substituted by a morpholinyl group substituted in the 4 position by the group R⁶, where R⁶ is as hereinbefore defined, and where the pyrrolidinyl, piperidinyl, piperazinyl and morpholinyl groups mentioned above in the definition of the group R⁶ may each be substituted by one or two C₁₋₃-alkyl groups, and where by the aryl groups mentioned in the definition of the foregoing groups is meant in each case a phenyl group which is mono- or disubstituted by R⁷,
where the substituents may be identical or different and R⁷ is a hydrogen atom, a fluorine, chlorine, bromine or iodine atom or a C₁₋₃-alkyl, hydroxy, C₁₋₃-alkyloxy, difluormethyl, trifluormethyl, difluoromethoxy, trifluormethoxy or cyano group; and
where by the heteroaryl groups mentioned in the definition of the foregoing groups is meant a pyridyl, pyridazinyl, pyrimidinyl or pyrazinyl group, where the above-mentioned heteroaryl groups are mono- or disubstituted by the group R⁷, where the substitutents may be the same or different and R⁷ is as hereinbefore defined; and
unless stated otherwise, the above-mentioned alkyl groups may be straight-chain or branched.

For example, compounds of the formula

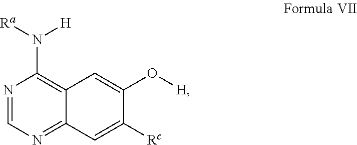

where R^{a} and R^{c} are as described in WO 2008/095847, are suitable for use. For example, as described in WO 2008/095847, R^{a} and R^{c} of Formula VII can be as described for R^{a} and R^{c} of Formula VI above.

As another example, compounds of the formula

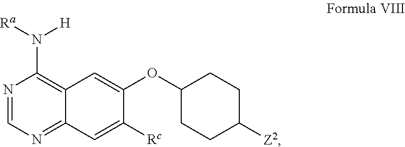

where R^{a}, R^{c}, and Z² are as described in WO 2008/095847, are suitable for use. For example, as described in WO 2008/095847, R^{a} and R^{c} of Formula VIII can be as described for R^{a} and R^{c} of Formula VI above, and Z² is a leaving group such as a halogen atom, e.g., a chlorine or bromine atom, or a sulfonyloxy group such as a methanesulfonyloxy or p-toluenesulphonyloxy group.

As another example, compounds of the formula

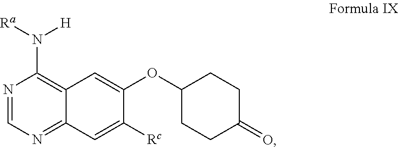

where R^{a} and R^{c} are as described in WO 2008/095847, are suitable for use. For example, as described in WO 2008/095847, R^{a} and R^{c} of Formula IX can be as described for R^{a} and R^{c} of Formula VI above.

As another example, compounds of the formula

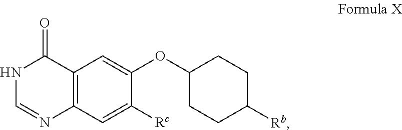

where R^{b} and R^{c} are as described in WO 2008/095847, are suitable for use. For example, as described in WO 2008/095847, R^{b} and R^{c} of Formula X can be as described for R^{b} and R^{c} of Formula VI above.

As another example, compounds of the formula

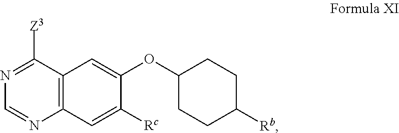

where R^{b}, R^{c}, and Z³ are as described in WO 2008/095847, are suitable for use. For example, as described in WO 2008/095847, R^{b} and R^{c} of Formula XI can be as described for R^{b} and R^{c} of Formula VI above, and Z³ is a halogen atom.

As another example, compounds of the formula

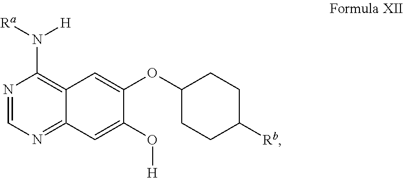

where R^{a} and R^{b} are as described in WO 2008/095847, are suitable for use. For example, as described in WO 2008/095847, R^{a} and R^{b} of Formula XII can be as described for R^{a} and R^{b} of Formula VI above.

As another example, compounds of the formula

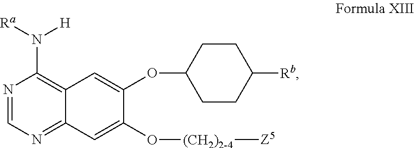

where R^{a}, R^{b}, and Z⁵ are as described in WO 2008/095847, are suitable for use. For example, as described in WO 2008/095847, R^{a} and R^{b} of Formula XIII can be as described for R^{a} and R^{b} of Formula VI above, and Z⁵ is a leaving group such as a halogen atom, e.g., a chlorine or bromine atom, or a sulfonyloxy group such as a methanesulfonyloxy or p-toluenesulphonyloxy group.

As another example, compounds of the formula

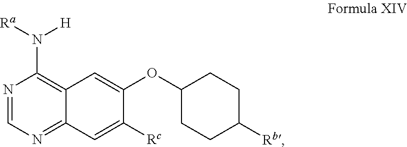

where R^{a}, R^{b′}, and R^{c} are as described in WO 2008/095847, are suitable for use. For example, as described in WO 2008/095847, R^{a} and R^{c} of Formula XIV can be as described for R^{a} and R^{c} of Formula VI above, and R^{b′} contains one or more groups that can be converted into hydroxyl groups, for example, an optionally substituted benzyloxy group, a silyloxy, acetyloxy, benzyloxy, methoxy, ethoxy, tert-butoxy or trtyloxy group.

As another example, compounds of the formula

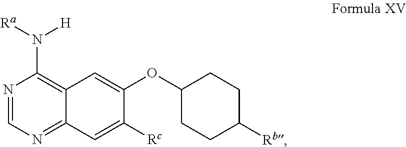

where R^{a}, R^{b″}, and R^{c} are as described in WO 2008/095847, are suitable for use. For example, as described in WO 2008/095847, R^{a} and R^{c} of Formula XV can be as described for R^{a} and R^{c} of Formula VI above, and R^{b″} contains a protected nitrogen atom. Conventional protecting groups for an amino, alkylamino or imino group include, for example, the formyl, acetyl, trifluoroacetyl, ethoxycarbonyl, tert-butoxycarbonyl, benzyloxycarbonyl, benzyl, methoxybenzyl or 2,4-dimethoxybenzyl group, while additionally the phthalyl group may be used for the amino group.

Exemplary suitable compounds include, e.g., 4-[(3-Chlor-2-fluor-phenyl)amino]-6-[cis-4-(morpholin-4-yl)-cyclohexyloxy]-7-methoxy-quinazoline; 4-[(3-Chlor-2-fluor-phenyl)amino]-6-[trans-4-(morpholin-4-yl)-cyclohexyloxy]-7-methoxy-quinazoline; 4-[(3-Chlor-2-fluor-phenyl)amino]-6-[(R)-cis-4-(3-hydroxy-pyrrolidin-1-yl)-cyclohexyloxy]-7-methoxy-quinazoline; 4-[(3-Chlor-2-fluor-phenyl)amino]-6-[(R)-trans-4-(3-hydroxy-pyrrolidin-1-yl)-cyclohexyloxy]-7-methoxy-quinazoline; 4-[(3-Chlor-2-fluor-phenyl)amino]-6-[(S)-cis-4-(3-hydroxy-pyrrolidin-1-yl)-cyclohexyloxy]-7-methoxy-quinazoline; 4-[(3-Chlor-2-fluor-phenyl)amino]-6-[(S)-trans-4-(3-hydroxy-pyrrolidin-1-yl)-cyclohexyloxy]-7-methoxy-quinazoline; 4-[(3-Chlor-2-fluor-phenyl)amino]-6-[cis-4-(3-oxo-piperazin-1-yl)-cyclohexyloxy]-7-methoxy-quinazoline; 4-[(3-Chlor-2-fluor-phenyl)amino]-6-[trans-4-(3-oxo-piperazin-1-yl)-cyclohexyloxy]-7-methoxy-quinazoline; 4-[(3-Chlor-2-fluor-phenyl)amino]-6-{(S)-cis-4-[2-(N,N-dinnethylanninocarbonyl)-pyrrolidin-i-yll-cyclohexyloxy-methoxy-quinazoline; 4-[(3-Chlor-2-fluor-phenyl)amino]-6-{(S)-trans-4-[2-(N,N-dimethylaminocarbonyl)-pyrrolidin-1-yl]-cyclohexyloxy]-7-methoxy-quinazoline; 4-[(3-Chlor-2-fluor-phenyl)amino]-6-{(S)-cis-4-[2-(anninocarbonyl)-pyrrolidin-1-yl]-cyclohexyloxy]-7-methoxy-quinazoline; 4-[(3-Chlor-2-fluor-phenyl)amino]-6-{(S)-trans-4-[2-(anninocarbonyl)-pyrrolidin-1-yl]-cyclohexyloxy]-7-methoxy-quinazoline; 4-[(3-Chlor-2-fluor-phenyl)amino]-6-[trans-4-(4-nnethyl-3-oxo-piperazin-1-yl)-cyclohexyloxy]-7-methoxy-quinazoline; 4-[(2-Fluor-3-methyl-phenyl)amino]-6-[trans-4-(3-oxo-piperazin-1-yl)-cyclohexyloxy]-7-methoxy-quinazoline; 4-[(2-Fluor-5-methyl-phenyl)amino]-6-[trans-4-(3-oxo-piperazin-1-yl)-cyclohexyloxy]-7-methoxy-quinazoline; 4-[(2,4-Difluor-3-methyl-phenyl)amino]-6-[trans-4-(3-oxo-piperazin-1-yl)-cyclohexyloxy]-7-methoxy-quinazoline; and 4-[(3-Chlor-2-methyl-phenyl)annino]-6-[trans-4-(3-oxo-piperazin-1-yl)-cyclohexyloxy]-7-methoxy-quinazoline.

Another suitable EGF-R inhibitor is EKB-569. EKB-569 is a 3-cyanoquinoline that irreversibly inhibits EGF-R tyrosine kinase activity. See, e.g., Erlichman et al. (2006) J. Clin. Oncol. 24:2252 for a description of EKB-569. EKB-569 is (N-[4-[(3-chloro-4-fluorophenyl)amino]-3-cyano-7-ethoxy-6-quinolinyl]-4-(dimethylamino)-2-butenamide).

Another suitable small molecule EGFR inhibitor is PD 183805 (CI 1033; 2-propenamide, N-[4-[(3-chloro-4-fluorophenyl)amino]-7-[3-(4-morpholinyl)propoxy]-6-quinazolinyl]-, dihydrochloride; Pfizer Inc.). CI-1033 is an orally available 4-anilinoquinazolone irreversible tyrosine kinase inhibitor.

Additional suitable small molecule EGFR inhibitors include, but are not limited to, ZM 105180 ((6-amino-4-(3-methylphenyl-amino)-quinazoline; Zeneca); BIBX-1382 (N-8-(3-chloro-4-fluoro-phenyl)-N-2-(1-methyl-piperidin-4-yl)-pyrimido[5,-4-d]pyrimidine-2,8-diamine; Boehringer Ingelheim); CL-387785 (N-[4-[(3-bromophenyl)amino]-6-quinazolinyl]-2-butynamide); BIBU1361 [(3-chloro-4-fluoro-phenyl)-[6-(4-diethylaminomethyl-piperidin-1-yl)-pyrimido[5,4-d]pyrimidin-4-yl]-amine]; or a salt of any of the foregoing. For example, BIBX-1382 dihydrochloride salt is suitable for use. See, e.g., Dittrich et al. (2002) J. Pharmacol. Exp. Ther. 311:502 for a description of BIBX-1382. See, e.g., Discafani et al. (1999) Biochem. Pharmacol. 57:917 for a description of CL-387785. See, e.g., Solca et al. (2004) J. Pharmacol. Expt'l Ther. 311:502 for a discussion of BIBU1361.

Additional suitable small molecule EGFR inhibitors include 6-furanylquinazoline. An example of such an inhibitor is GW572016 (Tykerb™; Lapatinib), an ErbB-2 and EGFR dual, reversible, tyrosine kinase inhibitor. GW572016 is a 6-furanylquinazoline. GW572016 is N-[3-chloro-4-[(3-fluorophenyl)methoxy]phenyl]-6-[5-[(2-methylsulfonylethylamino)methyl]furan-2-yl]quinazolin-4-amine.

Suitable EGF-R tyrosine kinase inhibitors also include, for example multi-kinase inhibitors that have activity on EGF-R kinase, i.e. inhibitors that inhibit EGF-R kinase and one or more additional kinases. Examples of such compounds include the EGF-R and HER2 inhibitor CI-1033 (formerly known as PD183805; Pfizer); the EGF-R and HER2 inhibitor GW-2016 (also known as GW-572016 or lapatinib ditosylate); the EGF-R and JAK 2/3 inhibitor AG490 (a tyrphostin); the EGF-R and HER2 inhibitor ARRY-334543 (4-dimethylamino-but-2-enoic acid; Array BioPharma); BIBW-2992, an irreversible dual EGF-R/HER2 kinase inhibitor (4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline; Boehringer Ingelheim Corp.); the EGF-R and HER2 inhibitor EKB-569 (Wyeth); the VEGF-R2 and EGFR inhibitor ZD6474 (ZACTIMA™; AstraZeneca Pharmaceuticals), and the EGF-R and HER2 inhibitor BMS-599626 (Bristol-Myers Squibb; see, e.g., Wong et al. (2006) Clin. Cancer Res. 12:6186 for the structure of BMX-599626).

Also suitable for use are pharmaceutically acceptable salts of any of the aforementioned EGF-R antagonists. Also suitable for use is a pro-drug of any of the aforementioned EGF-R antagonists. Also suitable for use are analogs and derivatives of any of the aforementioned EGF-R antagonists.

In certain embodiments, one or more of the aforementioned EGF-R antagonists is specifically excluded. In certain embodiments, one or more of the aforementioned classes of EGF-R antagonists is specifically excluded.

### Antibody Antagonists

Suitable EGF-R antagonists include antibodies that specifically bind EGF-R and inhibit the activity of the EGF-R, e.g., inhibit signal transduction activity, inhibit binding of an EGF-R ligand to EGF-R, etc.

Suitable antibody EGF-R antagonists include monoclonal antibodies (including neutralizing antibodies, chimerized, and humanized antibodies), antibody compositions with polyepitopic specificity, single-chain antibodies, immunoconjugates and fragments of antibodies. Anti-EGF-R antibodies can be of any isotype, e.g., IgG, including IgG subtypes (e.g., e.g., IgG1, IgG2, IgG3, IgG4, IgA, and IgA2); IgM; IgA; etc.

Suitable antibody EGF-R antagonists include antibody fragments, e.g., a portion of an intact antibody comprising the antigen-binding or variable region of the intact antibody. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins comprising an antibody fragment, recombinant proteins comprising an antibody fragment, and multispecific antibodies formed from antibody fragment(s).

“Humanized” forms of non-human (e.g. murine) antibodies are specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. Generally, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, or all of at least two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317.

“Chimeric” antibodies are immunoglobulins in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567 and Morrison et al, Proc. Natl. Acad. Sci. USA, 81:6851-6855 (1984)).

Antibody-based EGFR kinase inhibitors include any anti-EGFR antibody or antibody fragment that can partially or completely block EGFR activation by its natural ligand. Non-limiting examples of antibody-based EGF-R inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243; and U.S. Pat. No. 5,891,996. For example, the EGFR kinase inhibitor can be monoclonal antibody Mab E7.6.3 (Yang, X. D. et al. (1999) Cancer Res. 59:1236-43), or Mab C225 (ATCC Accession No. HB-8508; see, e.g., Petit et al. (1997) Am. J. Pathol. 151:1523; and Kawamoto et al. (1983) Proc. Natl. Acad. Sci. USA 80:1337), or an antibody or antibody fragment having the binding specificity thereof. Suitable monoclonal antibody EGFR kinase inhibitors include, but are not limited to, IMC-C225 (also known as cetuximab or ERBITUX™; Imclone Systems; see, e.g., WO 96/40210), ABX-EGF (Abgenix), EMD 72000 (Merck KgaA, Darmstadt), RH3 (York Medical Bioscience Inc.), and MDX-447 (Medarex/Merck KgaA), and EMD559900 (also known as MAb 425; see, e.g., Schnürch et al. (1994) Eur. J. Cancer 30A:491).

In certain embodiments, EGF-R antibody antagonists are specifically excluded. In certain embodiments, one or more specific EGF-R antibody antagonists are specifically excluded.

### Inhibitory Nucleic Acids

EGFR kinase inhibitors for use in a subject method can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in a subject method. EGFR gene expression can be reduced by contacting the tumor, subject or cell with a small double stranded RNA (dsRNA), or a vector or construct causing the production of a small double stranded RNA, such that expression of EGFR is specifically inhibited (e.g., RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836.

In some embodiments, an EGF-R inhibitor suitable for use in a subject method is an inhibitory (or “interfering”) nucleic acid. Interfering nucleic acids (RNAi) include nucleic acids that provide for decreased levels of an EGF-R polypeptide in a cell, e.g., a neuronal cell. Interfering nucleic acids include, e.g., a short interfering nucleic acid (siNA), a short interfering RNA (siRNA), a double-stranded RNA (dsRNA), a micro-RNA (miRNA), and a short hairpin RNA (shRNA) molecule.

The term “short interfering nucleic acid,” “siNA,” “short interfering RNA,” “siRNA,” “short interfering nucleic acid molecule,” “short interfering oligonucleotide molecule,” or “chemically-modified short interfering nucleic acid molecule” as used herein refers to any nucleic acid molecule capable of inhibiting or down regulating gene expression, for example by mediating RNA interference “RNAi” or gene silencing in a sequence-specific manner. Design of RNAi molecules when given a target gene is routine in the art. See also US 2005/0282188 (which is incorporated herein by reference) as well as references cited therein. See, e.g., Pushparaj et al. Clin Exp Pharmacol Physiol. 2006 May-June; 33(5-6):504-10; Lutzelberger et al. Handb Exp Pharmacol. 2006; (173):243-59; Aronin et al. Gene Ther. 2006 March; 13(6):509-16; Xie et al. Drug Discov Today. 2006 January; 11(1-2):67-73; Grunweller et al. Curr Med Chem. 2005; 12(26):3143-61; and Pekaraik et al. Brain Res Bull. 2005 Dec. 15; 68(1-2):115-20. Epub 2005 Sep. 9.

Methods for design and production of siRNAs to a desired target are known in the art, and their application to EGF-R-encoding nucleic acids will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

Publicly available tools to facilitate design of siRNAs are available in the art. See, e.g., DEQOR: Design and Quality Control of RNAi (available on the interne at cluster-1.mpi-cbg.de/Deqor/deqor.html). See also, Henschel et al. Nucleic Acids Res. 2004 Jul. 1; 32(Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand generally comprises nucleotide sequence that is complementary to nucleotide sequence in the other strand; such as where the antisense strand and sense strand form a duplex or double stranded structure, for example wherein the double stranded region is about 15 base pairs to about 30 base pairs, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 nucleotides to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post-transcriptional gene silencing, translational inhibition, or epigenetics. For example, siNA molecules of the invention can be used to epigenetically silence a target gene at the post-transcriptional level and/or at the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178). The multifunctional siNA can comprise sequence targeting, for example, two regions of Skp2.

siNA molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) that can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein), describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes include those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

In certain embodiments, EGF-R inhibitory nucleic acid antagonists are specifically excluded.

### Combination Therapy

As described in detail below, in some embodiments, a subject method involves administration of an EGF-R inhibitor as monotherapy, e.g., administration of EGF-R inhibitor only, without co-administration of any other therapeutic agent. In other embodiments, a subject treatment method is a combination therapy involving administration of: a) an EGF-R inhibitor; and b) at least one additional therapeutic agent, where the EGF-R inhibitor and the at least one additional therapeutic agent are administered in combined amounts that are effective to treat a viral infection. Suitable additional therapeutic agents are described below.

A subject combination therapy can involve: a) administration of an EGF-R inhibitor and at least one additional therapeutic agent at the same time, in the same formulation or in separate formulations; b) administration of at least one additional therapeutic agent within about 5 minutes to about 4 weeks of administration of an EGF-R inhibitor, e.g., administration of at least one additional therapeutic agent within about 5 minutes to about 15 minutes, within about 15 minutes to about 30 minutes, within about 30 minutes to about 60 minutes, within about 1 hour to about 2 hours, within about 2 hours to about 4 hours, within about 4 hours to about 8 hours, within about 8 hours to about 12 hours, within about 12 hours to about 24 hours, within about 24 hours to about 2 days, within about 2 days to about 4 days, within about 4 days to about 7 days, within about 1 week to about 2 weeks, or within about 2 weeks to about 4 weeks of administration of an EGF-R inhibitor.

In some embodiments, the at least one additional therapeutic agent is co-formulated with the EGF-R inhibitor. In other embodiments, the at least one additional therapeutic agent and the EGF-R inhibitor are separately formulated.

In some embodiments, an EGF-R inhibitor is administered for a first period of time, and an at least one additional therapeutic agent is administered for a second period of time, where the first period of time and the second period of time are overlapping. For example, in some embodiments, an EGF-R inhibitor is administered for a first period of time, and an at least one additional therapeutic agent is administered for a second period of time, where the second period of time begins before the end of the first period of time. an EGF-R inhibitor is administered for a first period of time, and an at least one additional therapeutic agent is administered for a second period of time, where the first period of time begins before the end of the second period of time. an EGF-R inhibitor is administered for a first period of time, and an at least one additional therapeutic agent is administered for a second period of time, where the first period of time begins before the beginning of the second period of time, and ends after the end of the second period of time.

In some embodiments, an effective amount of an EGF-R inhibitor and an at least one additional therapeutic agent are synergistic amounts. As used herein, a “synergistic combination” or a “synergistic amount” of EGF-R inhibitor and an additional (e.g., a second) therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the EGF-R inhibitor when administered at that same dosage as a monotherapy and (ii) the therapeutic or prophylactic benefit of the additional therapeutic agent when administered at the same dosage as a monotherapy.

### Viruses

Viral infections that can be treated with a subject method include infections of any of a variety of viruses, including, but not limited to, members of Picornaviridae; members of Orthomyxoviridae; members of Paramyxoviridae; members of Coronaviridae; members of Adenoviridae; members of Reoviridae; members of Caliciviridae; members of Astroviridae; members of Herpesviridae; members of Retroviridae; and members of Papillomaviridae.

As discussed below, certain viruses cause respiratory disorders in an infected individual. Such viruses are referred to herein as “respiratory viruses” and include, e.g., a rhinovirus, an influenza virus, a respiratory syncytial virus, a parainfluenza virus, a metapneumovirus, a coronavirus, an adenovirus, and other viruses noted below that can cause a respiratory disorder. In some embodiments, a subject method provides for treating a respiratory virus infection. In some embodiments, a subject method for treating a respiratory virus infection is a monotherapy and involves administering an effective amount of an EGF-R antagonist. In other embodiments, a subject method for treating a respiratory virus infection is a combination therapy and involves administering, in combined effective amounts: i) an EGF-R inhibitor; and ii) at least one additional therapeutic agent. Suitable additional therapeutic agents are discussed below. In some embodiments, the at least one additional therapeutic agent is an interferon (e.g., interferon-alpha, interferon-beta, interferon-gamma, interferon-lambda, interferon-tau, interferon-omega, etc.). In some embodiments, the at least one additional therapeutic agent is IFN-α. In some embodiments, the at least one additional therapeutic agent is IFN-β. In some embodiments, the at least one additional therapeutic agent is IFN-γ. In some embodiments, the at least one additional therapeutic agent is IFN-λ.

In some embodiments, a respiratory virus (e.g., a rhinovirus, an influenza virus, a respiratory syncytial virus, a parainfluenza virus, a metapneumovirus, a coronavirus, an adenovirus, etc.) causes exacerbation of a chronic lung disease (e.g., asthma, COPD, cystic fibrosis, emphysema, chronic bronchitis, interstitial lung disease, bronchitis; sarcoidosis, idiopathic pulmonary fibrosis, bronchiectasis, bronchiolitis, etc.). Thus, the present disclosure provides methods for treating respiratory virus-induced exacerbation of a chronic lung disease, where the methods involve administering an effective amount of an EGF-R antagonist in monotherapy, or administering, in combined effective amounts, an EGF-R antagonist and at least one additional therapeutic agent.

As discussed below, certain viruses cause gastrointestinal disorders in an infected individual. Such viruses are referred to herein as “gastrointestinal viruses” and include, e.g., an enterovirus, a hepatitis A virus, a rotavirus, a Norwalk virus, an astrovirus, or other virus discussed below that causes a gastrointestinal disorder in an infected individual.

In any of the above embodiments discussed below, the individual being treated using a subject method is a human of from about one month to about 6 months, from about 6 months to about 1 year, or from about 1 year to about 5 years of age. In any of the above embodiments discussed below, the individual being treated using a subject method is a human of from about 5 years to about 12 years, from about 13 years to about 18 years, or from about 18 years to about 25 years of age. In any of the above embodiments discussed below, the individual being treated using a subject method is a human of from about 25 years to about 50 years, from about 50 years to about 75 years of age, or older than 75 years of age. In any of the above embodiments discussed below, the individual being treated using a subject method is a human who is immunocompromised.

In some embodiments, a subject method provides for treating a gastrointestinal virus infection. In some embodiments, a subject method for treating a gastrointestinal virus infection is a monotherapy and involves administering an effective amount of an EGF-R antagonist. In other embodiments, a subject method for treating a gastrointestinal virus infection is a combination therapy and involves administering, in combined effective amounts: i) an EGF-R inhibitor; and ii) at least one additional therapeutic agent.

### Picornaviridae

The present disclosure provides methods for treating a Picornaviridae infection (also referred to as a “picornaviral infection”), e.g., an infection with a member of the Picornaviridae family. In general, a subject method for treating a picornaviral infection comprises administering an effective amount of an EGF-R inhibitor, as described above. The picornavirus infection may be caused by any virus of the family Picornaviridae. Representative family members include human rhinoviruses, polioviruses, enteroviruses including coxsackieviruses and echoviruses, hepatovirus, cardioviruses, apthovirus, hepatitis A and other picornaviruses not yet assigned to a particular genus, including one or more of the serotypes of these viruses.

In some embodiments, the viral infection is caused by a rhinovirus. In some embodiments, the rhinovirus is one that binds to intercellular adhesion molecule-1 (ICAM-1). Rhinoviruses that bind ICAM-1 belong to the “major group” of rhinoviruses; the major group includes about 91 serotypes. In some embodiments, the rhinovirus is one that binds to a low density lipoprotein (LDL) receptor (LDLR) family member. Rhinoviruses that bind an LDLR family member belong to the “minor group” of rhinoviruses; the minor group includes about 10 serotypes. For example, in some embodiments, the viral infection is caused by a major group human rhinovirus (HRV), e.g., HRV3, HRV14, HRV16, etc. In some embodiments, the viral infection is caused by a minor group HRV, e.g., HRV1A, HRV1B, HRV2, etc. See, e.g., Vlasak et al. (2003) J. Virol. 77:6923. In addition, human rhinoviruses have also been classified into three groups (A, B, C). In some embodiments, rhinovirus is a member of group A, B, or C. The present disclosure provides methods of treating a rhinovirus infection, e.g., a rhinovirus infection caused by a Group A rhinovirus, a Group B rhinovirus, or a Group C rhinovirus.

In some embodiments, the viral infection is caused by a coxsackievirus. Group A coxsackieviruses comprise about 23 serotypes, and cause diseases such as hand, foot, and mouth disease, and herpangina. For example, coxsackievirus A16 causes hand, foot, and mouth disease. Coxsackievirus A24 causes acute hemorrhagic conjunctivitis. Group B coxsackieviruses comprise about 6 serotypes, and causes diseases such as myocarditis, pleuritis, and pericarditis. Both Group A and B coxsackieviruses can cause aseptic meningitis. The present disclosure provides methods of treating a coxsackievirus infection.

In some embodiments, the viral infection is caused by hepatitis A virus. Hepatitis A virus can cause gastroenteritis and diarrhea.

In some embodiments, a subject method provides for treating a rhinovirus infection, the method involving administering to an individual in need thereof an effective amount of an EGF-R antagonist. In some embodiments, a subject method provides for treating a rhinovirus infection caused by a major group rhinovirus, the method involving administering to an individual in need thereof an effective amount of an EGF-R antagonist. In some embodiments, a subject method provides for treating a rhinovirus infection caused by a minor group rhinovirus, the method involving administering to an individual in need thereof an effective amount of an EGF-R antagonist. In some embodiments, a subject method provides for treating a rhinovirus infection caused by a member of rhinovirus group A, the method involving administering to an individual in need thereof an effective amount of an EGF-R antagonist. In some embodiments, a subject method provides for treating a rhinovirus infection caused by a member of rhinovirus group B, the method involving administering to an individual in need thereof an effective amount of an EGF-R antagonist. In some embodiments, a subject method provides for treating a rhinovirus infection caused by a member of rhinovirus group C, the method involving administering to an individual in need thereof an effective amount of an EGF-R antagonist.

In some embodiments, a subject method provides for treating a coxsackievirus infection, the method comprising administering an effective amount of an EGF-R antagonist to an individual having a coxsackievirus infection. In some embodiments, a subject method provides for treating hand, foot, and mouth disease caused by a coxsackievirus infection, the method comprising administering an effective amount of an EGF-R antagonist to an individual having hand, foot, and mouth disease. In some embodiments, a subject method provides for treating myocarditis, pleuritis, or pericarditis caused by a coxsackievirus infection, the method comprising administering an effective amount of an EGF-R antagonist to an individual having myocarditis, pleuritis, or pericarditis. In some embodiments, a subject method provides for treating meningitis caused by a coxsackievirus infection, the method comprising administering an effective amount of an EGF-R antagonist to an individual having meningitis. In some embodiments, a subject method provides for treating a hepatitis A virus infection, the method comprising administering an effective amount of an EGF-R antagonist to an individual having a hepatitis A virus infection.

In any of the above embodiments, the individual is a human of from about one month to about 6 months, from about 6 months to about 1 year, from about 1 year to about 5 years, from about 5 years to about 12 years, from about 13 years to about 18 years, from about 18 years to about 25 years, from about 25 years to about 50 years, from about 50 years to about 75 years of age, or older than 75 years of age. In any one of the above embodiments, the individual is a human who is immunocompromised. In some embodiments, the individual has a chronic lung disease (e.g., emphysema, COPD, chronic bronchitis, asthma, cystic fibrosis, bronchiectasis, bronchiolitis, or interstitial lung disease). In some embodiments, the individual has, in addition to a rhinovirus infection, pneumonia, where the pneumonia is caused by the rhinovirus or by a bacterial infection.

In some embodiments, a subject method of treating a rhinovirus infection comprises administering an EGF-R antagonist as monotherapy, e.g., where the EGF-R antagonist is the sole therapeutic agent being administered to the individual. In some embodiments, a subject method of treating rhinovirus infection is a combination therapy that comprises administering: a) an effective amount of an EGF-R antagonist; and b) at least one additional therapeutic agent. In some embodiments, the at least one additional therapeutic agent is an interferon (e.g., interferon-alpha, interferon-beta, interferon-gamma, interferon-lambda, interferon-tau, interferon-omega, etc.).

### Orthomyxoviridae

In some embodiments, the viral infection is caused by a member of Orthomyxoviridae, e.g., an influenza virus. A subject method is suitable for treating an infection caused by any of the three types of influenza viruses: A, B, and C. A subject method is suitable for treating an infection caused by any of a variety of subtypes of influenza A virus, e.g., influenza virus of any of a variety of combinations of hemagglutinin (HA) and neuraminidase (NA) variants. Subtypes of influenza A virus that can be treated using a subject method include H1N1, H1N2, H3N2, and H5N1 subtypes. Avian influenza A virus infections that can be treated with a subject method include infections with an avian influenza A virus of any one of the subtypes H5 and H7, including H5N1, H7N7, H9N2, H7N2, and H7N3 viruses. A subject method is suitable for treating an infection caused by any strain of an influenza A subtype, an influenza B virus subtype, or an influenza C virus. An infection caused by any subtype of influenza A H5, influenza A H7, and influenza A H9 can be treated using a subject method.

Influenza virus causes respiratory illness. At times of maximal illness, peak quantities of 10⁴ to 10⁷ infectious units/ml are detected. An influenza virus infection may extensively involve the alveoli, e.g., in patients with underlying heart or lung disease. Involvement of the alveoli may result in interstitial pneumonia, sometimes with marked accumulation of lung hemorrhage and edema fluid. Pure viral pneumonia of this type is a severe illness with a high mortality. Virus titers in secretions are high, and viral shedding is prolonged. In many instances, bacteria contribute to pneumonia in association with an influenza virus infection. Bacterial infection may occur before or after the viral infection. Examples of bacteria that can cause pneumonia associated with influenza virus infection include pneumococci, staphylococci, and Gram-negative bacteria.

In some embodiments, a subject method of treating an influenza virus infection involves administering to an individual having an influenza virus infection an effective amount of an EGF-R inhibitor. In some embodiments, a subject method of treating an influenza A virus infection involves administering to an individual having an influenza A virus infection an effective amount of an EGF-R inhibitor. In some embodiments, a subject method of treating an influenza B virus infection involves administering to an individual having an influenza B virus infection an effective amount of an EGF-R inhibitor. In some embodiments, the individual is an otherwise healthy individual. In some embodiments, the individual is a human of from about one month to about 6 months, from about 6 months to about 1 year, from about 1 year to about 5 years, from about 5 years to about 12 years, from about 13 years to about 18 years, from about 18 years to about 25 years, from about 25 years to about 50 years, from about 50 years to about 75 years of age, or older than 75 years of age. In any one of the above embodiments, the individual is a human who is immunocompromised. In some embodiments, the individual has a chronic lung disease (e.g., emphysema, chronic bronchitis, asthma, cystic fibrosis, bronchiectasis, or interstitial lung disease). In some embodiments, the individual has, in addition to an influenza virus infection, pneumonia, where the pneumonia is caused by the influenza virus or by a bacterial infection.

In some embodiments, a subject method of treating an influenza virus infection comprises administering an EGF-R antagonist as monotherapy, e.g., where the EGF-R antagonist is the sole therapeutic agent being administered to the individual. In some embodiments, a subject method of treating an influenza virus infection is a combination therapy that comprises administering: a) an effective amount of an EGF-R antagonist; and b) at least one additional therapeutic agent.

In some embodiments, the at least one additional therapeutic agent is a neuraminidase inhibitor, e.g., where the influenza virus is influenza A or influenza B. Suitable neuraminidase inhibitors include, e.g., oseltamivir (ethyl (3R,4R,5S)-5-amino-4-acetamido-3-(pentan-3-yloxy)cyclohex-1-ene-1-carboxylate; Tamiflu™), zanamivir (2R,3R,4S)-4-[diaminomethylidene)amino]-3-acetamido-2-[(1R,2R)-1,2,3-trihydroxypropyl]-3,4-dihydro-2H-pyran-6-carboxylic acid; Relenza™), and peramivir (1S,2S,3S,4R)-3-[(1S)-1-acetamido-2-ethyl-butyl]-4-(diaminomethylideneamino)-2-hydroxy-cyclopentane-1-carboxylic acid). In some embodiments, the at least one additional therapeutic agent is an M2 blocker, e.g., blocks a viral ion channel (M2 protein). The antiviral drugs amantadine and rimantadine are M2 blockers, and can be used in subject method of treating an influenza A virus infection. In some embodiments, the at least one additional therapeutic agent is an interferon (e.g., interferon-alpha, interferon-beta, interferon-gamma, interferon-lambda, interferon-tau, interferon-omega, etc.). In some embodiments, e.g., where the individual has an influenza virus infection and has pneumonia caused by a bacterial infection, the at least one additional therapeutic agent is an antibiotic that inhibits growth of the bacteria that caused the pneumonia.

### Paramyxoviridae

In other embodiments, the viral infection is caused by a member of Paramyxoviridae, e.g., respiratory syncytial virus, a human parainfluenza virus, rubulavirus (e.g., mumps virus), measles virus, and human metapneumovirus.

Measles virus causes a disease marked by a prodrome of fever, conjunctivitis, coryza, and cough, followed by the development of a rash of flat macules, which first appear on the head, then move to the trunk and limbs. Two serious complications of measles virus infection are acute postinfectious encephalitis, and subacute sclerosing panencephalitis.

In some embodiments, the viral infection is caused by a parainfluenza virus of any known type, e.g., any one of types 1, 2, 3, 4a, and 4b. Parainfluenza virus is a common respiratory pathogen of humans. Parainfluenza viruses are the most common cause of croup or laryngotracheobronchitis, in children aged 6 months to 5 years. Parainfluenza viruses are also capable of causing bronchiolitis and/or pneumonia in children under the age of 6 months. Parainfluenza virus is also responsible for viral pneumonia in adults, and can cause exacerbations of chronic lung diseases (e.g., asthma, chronic obstructive pulmonary disease (COPD), bronchiectasis, and cystic fibrosis).

In some embodiments, the viral infection is caused by a respiratory syncytial virus. Respiratory syncytial virus (RSV) is an important pathogen of infants. RSV can present as a febrile rhinitis and/or pharyngitis and often involves the middle ear. RSV is the most common cause of bronchiolitis in children and also causes pneumonia in adults. In addition, RSV is a cause of exacerbations of chronic lung diseases (e.g., asthma, COPD, bronchiectasis, and cystic fibrosis).

Human metapneumovirus accounts for approximately 10% of respiratory tract infections that are not related to any previously known etiologic agent. Human metapneumovirus can cause mild respiratory tract infection; however small children, elderly individuals, and immunocompromised individuals are at risk of severe disease and hospitalization. In addition, human metapneumovirus is a cause of exacerbations of chronic lung diseases (e.g., asthma, COPD, bronchiectasis, and cystic fibrosis).

In some embodiments, a subject method involves administering an effective amount of an EGF-R antagonist to an individual infected with a member of the Paramyxoviridae family. In some embodiments, a subject method involves administering an effective amount of an EGF-R antagonist to an individual infected with a respiratory syncytial virus. In some embodiments, a subject method involves administering an effective amount of an EGF-R antagonist to an individual infected with a measles virus. In some embodiments, a subject method involves administering an effective amount of an EGF-R antagonist to an individual infected with parainfluenza virus (e.g., a virus of any one of types 1, 2, 3, 4a, and 4b). In some embodiments, a subject method involves administering an effective amount of an EGF-R antagonist to an individual infected with a human metapneumovirus. In any one of the above embodiments, the individual is a human of from about one month to about 6 months, from about 6 months to about 1 year, from about 1 year to about 5 years, from about 5 years to about 12 years, from about 13 years to about 18 years, from about 18 years to about 25 years, from about 25 years to about 50 years, from about 50 years to about 75 years of age, or older than 75 years of age. In any one of the above embodiments, the individual is a human who is immunocompromised. In some embodiments, the individual has a chronic lung disease (e.g., emphysema, chronic bronchitis, asthma, cystic fibrosis, bronchiectasis, bronchiolitis, COPD, or interstitial lung disease). In some embodiments, the individual has, in addition to a virus infection (e.g., a viral infection caused by a parainfluenza virus, an RSV, or a human metapneumonia virus), pneumonia, where the pneumonia is caused by the virus (e.g., a parainfluenza virus, an RSV, or a human metapneumonia virus) or by a bacterial infection.

In some embodiments, a subject method of treating a virus infection, where the virus is a member of the family Paramyxoviridae, comprises administering an EGF-R antagonist as monotherapy, e.g., where the EGF-R antagonist is the sole therapeutic agent being administered to the individual. In some embodiments, a subject method of treating a virus infection, where the virus is a member of the family Paramyxoviridae, is a combination therapy that comprises administering: a) an effective amount of an EGF-R antagonist; and b) at least one additional therapeutic agent. In some embodiments, the at least one additional therapeutic agent is an interferon (e.g., interferon-alpha, interferon-beta, interferon-gamma, interferon-lambda, interferon-tau, interferon-omega, etc.).

In some embodiments, the at least one additional therapeutic agent is ribavirin or a ribavirin derivative. Ribavirin, 1-β-D-ribofuranosyl-1H-1,2,4-triazole-3-carboxamide, is described in the Merck Index, compound No. 8199, Eleventh Edition. Its manufacture and formulation is described in U.S. Pat. No. 4,211,771. Also suitable for use are derivatives of ribavirin (see, e.g., U.S. Pat. No. 6,277,830). The ribavirin can be administered orally in capsule or tablet form, or in the same or different administration form and in the same or different route as the EGF-R inhibitor. Other routes/modes of administration of both medicaments are contemplated, such as by nasal spray, transdermally, by suppository, by sustained release dosage form, etc. Any form of administration will work so long as the proper dosages are delivered without destroying the active ingredient.

Ribavirin can be administered in an amount ranging from about 400 mg to about 1200 mg, from about 600 mg to about 1000 mg, or from about 700 to about 900 mg per day. In some embodiments, ribavirin is administered throughout the entire course of EGF-R inhibitor therapy. In other embodiments, ribavirin is administered only during the first period of time. In still other embodiments, ribavirin is administered only during the second period of time.

### Coronaviridae

The present disclosure provide methods for treating a virus infection, where the virus is a member of the Coronaviridae family, the method involving administering an effective amount of an EGF-R antagonist to an individual infected with a member of the Coronaviridae family. Coronaviridae includes, e.g., coronaviruses, e.g., human coronavirus 229E (HCoV-229E), human coronavirus OC43 (HCoV-OC43), and SARS-CoV (the causative agent of severe acute respiratory syndrome (SARS)), which cause upper respiratory tract infection, lower respiratory tract infections, and gastroenteritis.

In some embodiments, a method of treating a virus infection is provided, where the virus is a coronavirus, the method involving administering an effective amount of an EGF-R antagonist to an individual infected with a coronavirus, e.g., a coronavirus that infects a human (HCoV). In some embodiments, a method of treating a virus infection is provided, where the virus is a Group 1 coronavirus, the method involving administering an effective amount of an EGF-R antagonist to an individual infected with a Group 1 coronavirus. In some embodiments, a method of treating a virus infection is provided, where the virus is a Group 2 coronavirus, the method involving administering an effective amount of an EGF-R antagonist to an individual infected with a Group 2 coronavirus. In some embodiments, a method of treating a virus infection is provided, where the virus is a Group 3 coronavirus, the method involving administering an effective amount of an EGF-R antagonist to an individual infected with a Group 3 coronavirus. In some embodiments, a method of treating a virus infection is provided, where the virus is HCoV-OC43, the method involving administering an effective amount of an EGF-R antagonist to an individual infected with HCoV-OC43. In some embodiments, a method of treating a virus infection is provided, where the virus is HCoV-229E, the method involving administering an effective amount of an EGF-R antagonist to an individual infected with HCoV-229E. In some embodiments, a method of treating a virus infection is provided, where the virus is SARS-CoV, the method involving administering an effective amount of an EGF-R antagonist to an individual infected with SARS-CoV.

In any one of the above embodiments, the individual is a human of from about one month to about 6 months, from about 6 months to about 1 year, from about 1 year to about 5 years, from about 5 years to about 12 years, from about 13 years to about 18 years, from about 18 years to about 25 years, from about 25 years to about 50 years, from about 50 years to about 75 years of age, or older than 75 years of age. In some embodiments, the individual has a chronic lung disease (e.g., emphysema, chronic bronchitis, asthma, cystic fibrosis, bronchiectasis, COPD, or interstitial lung disease). In some embodiments, the individual has, in addition to a coronavirus infection, pneumonia, where the pneumonia is caused by the coronavirus or by a bacterial infection.

### Adenoviridae

The present disclosure provides methods of treating a virus infection in an individual, where the virus is a member of the Adenoviridae family, the methods generally involving administering an effective amount of an EGF-R antagonist to the individual. Members of the Adenoviridae include human adenovirus (HAdV)-A, HAdV-B, HAdV-C, HAdV-D, HAdV-E, and HAdV-F.

HAdV-B and HAdV-C can cause respiratory disease. HAdV-B and HAdV-D can cause conjunctivitis. HAdV-F serotypes 40 and 41 can cause gastroenteritis. Adenovirus can also cause bronchiolitis or pneumonia. Adenovirus can also cause viral meningitis or encephalitis.

In some embodiments, a subject method provides for treating a virus infection, where the virus is a member of the Adenoviridae family, the method generally involving administering an effective amount of an EGF-R antagonist to an individual infected with a member of the Adenoviridae family. In some embodiments, a subject method provides for treating an adenovirus infection in an individual, where the adenovirus is one of HAdV-A, HAdV-B, HAdV-C, HAdV-D, HAdV-E, and HAdV-F, the method generally involving administering an effective amount of an EGF-R antagonist to the individual. In some embodiments, a subject method provides for treating a respiratory disease caused by an HAdV infection (e.g., HAdV-B or HAdV-C), the methods generally involving administering an effective amount of an EGF-R antagonist to an individual having a respiratory disease caused by an HAdV infection (e.g., HAdV-B or HAdV-C). In some embodiments, a subject method provides for treating gastroenteritis caused by an HAdV infection (e.g., HAdV-F serotype 40 or 41), the methods generally involving administering an effective amount of an EGF-R antagonist to an individual having gastroenteritis caused by an HAdV infection (e.g., HAdV-F serotype 40 or 41). In some embodiments, a subject method provides for treating viral meningitis or encephalitis caused by an HAdV infection, the methods generally involving administering an effective amount of an EGF-R antagonist to an individual having viral meningitis or encephalitis caused by an HAdV infection.

In any one of the above embodiments, the individual is a human of from about one month to about 6 months, from about 6 months to about 1 year, from about 1 year to about 5 years, from about 5 years to about 12 years, from about 13 years to about 18 years, from about 18 years to about 25 years, from about 25 years to about 50 years, from about 50 years to about 75 years of age, or older than 75 years of age. In any one of the above embodiments, the individual is a human who is immunocompromised. In some embodiments, the individual has a chronic lung disease (e.g., emphysema, chronic bronchitis, asthma, cystic fibrosis, bronchiectasis, COPD, or interstitial lung disease). In some embodiments, the individual has, in addition to an adenovirus infection, pneumonia, where the pneumonia is caused by the adenovirus infection or by a bacterial infection.

### Reoviridae

In other embodiments, the viral infection is caused by a member of Reoviridae, e.g., a rotavirus. In some embodiments, the viral infection is caused by one of rotavirus-A, rotavirus-B, rotavirus-C, rotavirus-D, rotavirus-E, rotavirus-F, and rotavirus-G. In some embodiments, the viral infection is caused by rotavirus-A. Rotavirus-A causes about 90% of rotavirus infections in humans, and can cause severe diarrhea in infants and young children. Rotavirus gastroenteritis is a mild to severe disease characterized by vomiting, watery diarrhea, and low-grade fever.

In some embodiments, a subject method provides for treatment of a viral infection caused by a member of the Reoviridae family, the method generally involving administering an effective amount of an EGF-R antagonist to an individual infected with a member of the Reoviridae family. In some embodiments, a subject method provides for treatment of a viral infection caused by a rotavirus, the method generally involving administering an effective amount of an EGF-R antagonist to an individual infected with a rotavirus. In some embodiments, a subject method provides for treatment of a viral infection caused by rotavirus-A, the method generally involving administering an effective amount of an EGF-R antagonist to an individual infected with rotavirus-A. In some embodiments, a subject method provides for treating diarrhea in an individual, where the diarrhea is caused by a rotavirus-A infection, the method generally involving administering an effective amount of an EGF-R antagonist to the individual

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age. In any one of the above embodiments, the individual is a human who is immunocompromised.

### Caliciviridae

In some embodiments, the virus infection is caused by a member of the Caliciviridae family. Caliciviridae family members include, e.g., the genus calicivirus, which includes Norwalk virus. Norwalk virus causes gastroenteritis.

In some embodiments, a subject method provides for treatment of a viral infection caused by a member of the Caliciviridae family, the method generally involving administering an effective amount of an EGF-R antagonist to an individual infected with a member of the Caliciviridae family. In some embodiments, a subject method provides for treatment of a Norwalk virus infection in an individual, the method involving administering to the individual an effective amount of an EGF-R antagonist. In some embodiments, a subject method provides for treating gastroenteritis caused by a Norwalk virus infection, the method involving administering an effective amount of an EGF-R antagonist to an individual having gastroenteritis resulting from a Norwalk virus infection.

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age. In any one of the above embodiments, the individual is a human who is immunocompromised.

### Astroviridae

The present disclosure provides methods of treating a viral infection, where the virus is a member of the Astroviridae family, the methods comprising administering an effective amount of an EGF-R antagonist to an individual having a viral infection caused by a member of the Astroviridae family. The Astroviridae family includes the genus Mamastrovirus, members of which infect mammals, and includes human astrovirus; and the genus Avastroviruses, members of which infect birds. Human astrovirus is a cause of gastroenteritis in children and adults. The main symptoms are diarrhea, followed by nausea, vomiting, fever, malaise and abdominal pain.

In some embodiments, a subject method provides for treating a viral infection caused by a member of the Astroviridae family, the method generally involving administering an effective amount of an EGF-R antagonist to an individual having a viral infection caused by a member of the Astroviridae family. In some embodiments, a subject method provides for treating a human astrovirus infection in an individual, the method generally involving administering to the individual an effective amount of an EGF-R antagonist. In some embodiments, a subject method provides for treating gastroenteritis caused by a human astrovirus infection, the method generally involving an effective amount of an EGF-R antagonist to an individual having gastroenteritis caused by a human astrovirus infection.

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age. In any one of the above embodiments, the individual is a human who is immunocompromised.

### Retroviridae

In other embodiments, the viral infection is caused by a member of Retroviridae, e.g., a retrovirus such as a lentivirus. The term “retrovirus” is well understood in the art, and includes single-stranded, positive sense, enveloped RNA viruses that include, e.g., the genus Gammaretrovirus (e.g., murine mammary tumor virus); the genus Epsilonretrovirus; the genus Alpharetrovirus (e.g., avian leukosis virus); the genus Betaretrovirus; the genus Deltaretrovirus (e.g., bovine leukemia virus; human T-lymphotrophic virus (HTLV)); the genus Lentivirus; and the genus Spumavirus. Lentivirus is a genus of viruses of the Retroviridae family, and includes human immunodeficiency virus-1 (HIV-1); human immunodeficiency virus-2 (HIV-2); simian immunodeficiency virus. (SIV); and feline immunodeficiency virus (FIV).

In some embodiments, a subject method provides for treating a viral infection caused by a member of the Retroviridae family, the method generally involving administering an effective amount of an EGF-R antagonist to an individual having a viral infection caused by a member of the Retroviridae family. In some embodiments, a subject method provides for treating a retrovirus infection in an individual, the method generally involving administering an effective amount of an EGF-R antagonist to the individual. In some embodiments, a subject method provides for treating a lentivirus infection in an individual, the method generally involving administering an effective amount of an EGF-R antagonist to the individual. In some embodiments, a subject method provides for treating an HIV-1 infection in an individual, the method generally involving administering an effective amount of an EGF-R antagonist to the individual.

A subject method of treating a lentivirus infection is suitable treating individuals who have a human immunodeficiency virus (HIV) infection; individuals who are naïve with respect to HIV infection, but who at risk of contracting an HIV infection; and individuals who were treated for an HIV infection, but who either failed to respond to the treatment, or who initially responded to treatment but subsequently relapsed. Such individuals include, but are not limited to, uninfected individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals; intravenous drug users; individuals in whom a mucosal tissue may have been exposed to HIV-infected blood, blood products, or other HIV-contaminated body fluids; and babies who are being nursed by HIV-infected mothers. Individuals suitable for treatment include individuals infected with, or at risk of becoming infected with, HIV-1 and/or HIV-2 and/or HIV-3, or any variant thereof. Individuals suitable for treatment include any individual having mucosal exposure to HIV.

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age.

In some embodiments, a subject method of treating a viral infection caused by a member of the Retroviridae family involves administering an effective amount of an EGF-R antagonist as monotherapy. In other embodiments, a subject method of treating a viral infection caused by a member of the Retroviridae family is a combination therapy that involves administering i) an effective amount of an EGF-R antagonist; and ii) at least one additional therapeutic agent.

The at least one additional therapeutic agent can be a therapeutic agent for the treatment of a retroviral, e.g., a lentiviral infection, or for the treatment of a disorder that may accompany a retroviral, e.g., a lentiviral infection (e.g., a bacterial infection, a fungal infection, and the like). Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, an interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. In some embodiments, the at least one additional therapeutic agent is an interferon (e.g., interferon-alpha, interferon-beta, interferon-gamma, interferon-lambda, interferon-tau, interferon-omega, etc.).

### Herpesviridae

The present disclosure provides methods of treating a viral infection, where the virus is a member of the Herpesviridae family. The Herpesviridae family includes the sub-families Alphaherpesvirinae, Betaherpesvirinae, and Gammaherpesvirinae. The sub-family Alphaherpesvirinae includes the genera simplex virus (e.g., herpes simplex virus-1 (also knows as human herpesvirus-1 or HHV-1); and herpes simplex virus-2 (also known as human herpesvirus-2 or HHV-2)); and varicellovirus (e.g., Varicella Zoster Virus (VSV); also knows as human herpesvirus-3 or HHV-3). The sub-family Betaherpesvirinae includes the genera cytomegalovirus (CMV; also known as human herpesvirus-5 or HHV-5); and roseolovirus (also known as human herpesvirus-6 or HHV-6). The sub-family Gammaherpesvirinae includes the genera lymphocryptovirus (Epstein-Barr Virus (EBV); human herpesvirus-4 or HHV-4); and rhadinovirus (Kaposi's sarcoma-associated herpesvirus (KSHV); human herpesvirus-8 or HHV-8).

HSV-1 and HSV-2 infections are characterized by cold sores of skin, mouth or genital region. After primary infection, the virus is harbored in neural cells and can reappear later in the life of a patient. EBV causes infectious mononucleosis and it is considered as the etiologic agent of nasopharyngeal cancer, immunoblastic lymphoma, Burkitt's lymphoma and hairy leukoplakia. VZV causes chicken pox and shingles. Although in children the chicken pox is usually a non-fatal disease, the recurrent form of this infection, shingles, may in advanced stage lead to paralysis, convulsions, and ultimately death. Again, in immunocompromised patients the infection with VZV is a serious complication. Human herpes virus 6 (HHV-6) which is commonly associated with children's rash has also been identified in acquired immunodeficiency syndrome (AIDS) patients and it may be a cofactor in the pathogenesis of AIDS in hosts infected with human immunodeficiency virus (HIV).

In some embodiments, a subject method provides for treating a viral infection, where the virus is a member of the Herpesviridae family, the method generally involving administering an effective amount of an EGF-R antagonist to an individual infected with a member of the Herpesviridae family. In some embodiments, a subject method provides for treating an HSV-1 infection in an individual, the method comprising administering to the individual an effective amount of an EGF-R antagonist. In some embodiments, a subject method provides for treating an HSV-2 infection in an individual, the method comprising administering to the individual an effective amount of an EGF-R antagonist. In some embodiments, a subject method provides for treating a VSV infection in an individual, the method comprising administering to the individual an effective amount of an EGF-R antagonist. In some embodiments, a subject method provides for treating an EBV infection in an individual, the method comprising administering to the individual an effective amount of an EGF-R antagonist. In some embodiments, a subject method provides for treating an HHV-8 infection in an individual, the method comprising administering to the individual an effective amount of an EGF-R antagonist.

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age. In any one of the above embodiments, the individual is immunocompromised.

In some embodiments, a subject method of treating a viral infection caused by a member of the Herpesviridae family involves administering an effective amount of an EGF-R antagonist as monotherapy. In other embodiments, a subject method of treating a viral infection caused by a member of the Herpesviridae family is a combination therapy that involves administering i) an effective amount of an EGF-R antagonist; and ii) at least one additional therapeutic agent.

Suitable additional therapeutic agents, e.g., for the treatment of an HSV-1 or an HSV-2 infection include, but are not limited to, acyclovir (Zovirax), valganciclovir, famciclovir, valacyclovir (Valtrex), ganciclovir (Cytovene), cidofovir (Vistide), antisense oligonucleotide fomivirsen (Vitravene), foscarnet (Foscavir), penciclovir, idoxuridine, vidarabine, and trifluridine. In some embodiments, the at least one additional therapeutic agent is an interferon (e.g., interferon-alpha, interferon-beta, interferon-gamma, interferon-lambda, interferon-tau, interferon-omega, etc.).

Acyclovir is a purine nucleoside analog that can be used in a subject combination therapy for treating HSV-1, HSV-2, VZV, or EBV infection. Valacyclovir can be used in a subject combination therapy for treating HSV-1, HSV-2, VZV, or EBV infection. Cidofovir is a nucleotide analog that can be used in a subject combination therapy for treating HSV-1, HSV-2, VZV, EBV, or KSHV infection. Famciclovir is a prodrug that can be used in a subject combination therapy for treating HSV-1, HSV-2, or VZV infection. Foscarnet is an organic analog of inorganic pyrophosphate that can be used in a subject combination therapy for treating EBV, KSHV, HSV, or VZV infection. Ganciclovir is a nucleoside analog of 2′-deoxyguanosine that can be used in a subject combination therapy for treating any human herpesvirus (HHV) infection. Valganciclovir is an orally bioavailable form of ganciclovir that can be used in a subject combination therapy for treating any HHV infection. Idoxuridine can be used topically in a subject combination therapy to treat herpes simplex keratoconjunctivitis. Penciclovir is a phosphorylated guanosine analog that can be applied topically in a subject combination therapy to treat recurrent herpes labialis (e.g., caused by HSV-1 or HSV-2). Trifluridine is a thymine analog that can be used in a subject combination therapy for treating primary keratoconjunctivitis and recurrent keratitis or ulceration caused by HSV-1 and HSV-2. Vidarabine is an adenine arabinoside that can be used in a subject combination therapy for treating HSV-1 or HSV-2 infection.

Suitable routes of administration of the aforementioned additional therapeutic agents are known in the art. For example, ganciclovir is available as an oral formulation; cidofovir and fomivirsen are approved for topical application against retinitis in AIDS patients; and foscarnet is formulated for use by an intravenous route.

### Papillomaviridae

The present disclosure provides a method of treating a viral infection, where the virus is a member of the Papillomaviridae family, the method generally involving administering an effective amount of an EGF-R antagonist to an individual infected with a member of the Papillomaviridae family. Members of the Papillomaviridae family include human papillomaviruses that are members of the Alphapapillomavirus genus, the Betapapillomavirus genus, the Gammapapillomavirus genus, the Mupapillomavirus genus, and the Nupapillomavirus genus. Human papillomavirus (HPV) includes about 130 serotypes.

Members of the genus Alphapapillomavirus preferentially infect the oral or anogenital mucosa in humans and primates. Certain species (eg. Human papillomavirus 2, Human papillomavirus 10) are also found in lesions of cutaneous sites. Specific species (eg. Human papillomavirus 16, Human papillomavirus 18) are considered as high-risk virus in view of their regular presence in malignant tissue and their in vitro transforming activities. Other species (eg. Human papillomavirus 53, Human papillomavirus 26, Human papillomavirus 34) cause malignant or benign lesions, whereas the low-risk species (Human papillomavirus 61, Human papillomavirus 7, Human papillomavirus 6, Human papillomavirus 54, Human papillomavirus cand90, Human papillomavirus 71) mainly cause benign lesions.

Members of the genus Betapapillomavirus preferentially infect the skin of humans. These infections exist latent in the general population, but are activated under conditions of immunosuppression. Species Human papillomavirus 5, Human papillomavirus 9 and Human papillomavirus 49 are also associated with the disease Epidermodysplasia verruciformis (EV).

Members of the genus Gammapapillomavirus (e.g., Human papillomavirus-4) cause cutaneous lesions in their host. Mupapillomavirus (e.g., Human papillomavirus-1; Human papillomavirus-63) cause cutaneous lesions in their host. Nupapillomavirus (e.g., Human papillomavirus-41) cause benign and malignant cutaneous lesions in their hosts.

HPV types 16, 18, 31, 33, 35, 39, 45, 51, 52, 56, 58, 59, and 68 are considered sexually transmitted HPVs and can lead to the development of cervical intraepithelial neoplasia (CIN), vulvar intraepithelial neoplasia (VIN), penile intraepithelial neoplasia (PIN), and/or anal intraepithelial neoplasia (AIN). HPV-2 and HPV-7 can cause common cutaneous warts. HPV types 1, 2, and 4 can cause plantar warts. HPV types 6, 11, 42, 43, 44, 55 can cause anogenital warts. HPV types 6, 7, 11, 16, 32 can cause oral papillomas.

A subject method provides for treating a viral infection, where the virus is a member of the Papillomaviridae, the method comprising administering to an individual in need thereof an effective amount of an EGF-R antagonist. In some embodiments, a subject method provides for treating a human papillomavirus (HPV) infection, the method comprising administering an effective amount of an EGF-R antagonist to an individual infected with an HPV. In some embodiments, a subject method provides for treating an HPV infection, the method comprising administering an effective amount of an EGF-R antagonist to an individual infected with HPV type 16, 18, 31, 33, 35, 39, 45, 51, 52, 56, 58, 59, or 68. In some embodiments, a subject method provides for treating an HPV infection, the method comprising administering an effective amount of an EGF-R antagonist to an individual infected with HPV type 16. In some embodiments, a subject method provides for treating an HPV infection, the method comprising administering an effective amount of an EGF-R antagonist to an individual infected with HPV type 1, 2, or 4. In some embodiments, a subject method provides for treating an HPV infection, the method comprising administering an effective amount of an EGF-R antagonist to an individual infected with HPV type 2 or 7. In some embodiments, a subject method provides for treating an HPV infection, the method comprising administering an effective amount of an EGF-R antagonist to an individual infected with HPV type 6, 11, 42, 43, 44, or 55.

In any one of the above embodiments, the individual is a human of from about one month to about 6 months, from about 6 months to about 1 year, or from about 1 year to about 5 years of age. In any one of the above embodiments, the individual is a human of from about 5 years to about 12 years, from about 13 years to about 18 years, or from about 18 years to about 25 years of age. In any one of the above embodiments, the individual is a human of from about 25 years to about 50 years, from about 50 years to about 75 years of age, or older than 75 years of age. In any one of the above embodiments, the individual is immunocompromised.

In some embodiments, a subject method of treating a viral infection caused by a member of the Papillomaviridae family involves administering an effective amount of an EGF-R antagonist as monotherapy. In other embodiments, a subject method of treating a viral infection caused by a member of the Papillomaviridae family is a combination therapy that involves administering i) an effective amount of an EGF-R antagonist; and ii) at least one additional therapeutic agent. In some embodiments, the at least one additional therapeutic agent is an interferon (e.g., interferon-alpha, interferon-beta, interferon-gamma, interferon-lambda, interferon-tau, interferon-omega, etc.).

### Interferons

As noted above, the present disclosure contemplates, in some embodiments, the use of combination therapy to treat a viral infection, where the combination therapy involves administering i) an effective amount of an EGF-R antagonist; and ii) at least one additional therapeutic agent. As noted above, in some embodiments, the at least one additional is an interferon. Suitable interferons include, e.g., interferon-alpha (IFN-α), interferon-beta (IFN-β), interferon-gamma (IFN-γ), interferon-lambda (IFN-λ), IFN-tau, IFN-ω, etc.

### IFN-α

Any known IFN-α can be used in a subject combination therapy. The term “IFN-α” includes biologically active IFN-α, where biologically active IFN-α includes naturally occurring IFN-α; synthetic IFN-α; derivatized IFN-α (e.g., PEGylated IFN-α, glycosylated IFN-α, and the like); glycosylated IFN-α; IFN-α derivatized with poly(ethylene glycol) (“PEGylated IFN-α”); and analogs of naturally occurring or synthetic IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1).

Also suitable for use in a subject combination therapy are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548).

The term “IFN-α” also encompasses derivatives of IFN-α that are derivatized (e.g., are chemically modified) to alter certain properties such as serum half-life. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha-2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

### IFN-β

The term interferon-beta (“IFN-β”) includes biologically active IFN-β polypeptides that are naturally occurring; non-naturally-occurring IFN-β polypeptides; and analogs and variants of naturally occurring or non-naturally occurring IFN-β.

Any of a variety of beta interferons can be used in a subject treatment method. Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like. The IFN-β formulation may comprise an N-blocked species, wherein the N-terminal amino acid is acylated with an acyl group, such as a formyl group, an acetyl group, a malonyl group, and the like. Also suitable for use is a consensus IFN-β.

### IFN-γ

The term interferon-beta (“IFN-γ”) includes biologically active IFN-γ polypeptides that are naturally occurring; non-naturally-occurring IFN-γ polypeptides; and analogs and variants of naturally occurring or non-naturally occurring IFN-γ.

IFN-γ1b (Actimmune®; human interferon) is a single-chain polypeptide of 140 amino acids and is suitable for use in a subject combination therapy. Actimmune® is made recombinantly in E. coli and is unglycosylated (Rinderknecht et al. 1984, J. Biol. Chem. 259:6790-6797). Recombinant IFN-gamma as discussed in U.S. Pat. No. 6,497,871 is also suitable for use. Additional suitable IFN-γ forms are found in, e.g., U.S. Pat. No. 5,690,925; WO 01/36001; and WO 02/081507.

### IFN-λ

The term “IFN-λ” includes, e.g., IFN-λ1, IFN-λ2 and IFN-λ3. IFN-λ1 is also known as IL-29, while IFN-λ2 and IFN-λ3 are known as IL-28a/b. Amino acid sequences of IFN-λ1, IFN-λ2 and IFN-λ3 are known. See, e.g., SEQ ID NOs:5, 6, and 7 (IFN-λ1, IFN-λ2 and IFN-λ3, respectively) of US Patent Publication No. 2007/0134763. The IFN-λ polypeptide can have the same amino acid sequence as one of SEQ ID NOs:5, 6, or 7 of US Patent Publication No. 2007/0134763, or can have an amino acid sequence that is at least about 80%, at least about 85%, at least about 90%, at least about 95%, or at least about 98%, identical to one of SEQ ID NOs:5, 6, or 7 of US Patent Publication No. 2007/0134763, where the IFN-λ polypeptide is biologically active. The term “IFN-λ” includes naturally-occurring IFN-λ, recombinant IFN-λ, and synthetic IFN-λ. The term “IFN-λ” also includes modified IFN-λ, e.g., PEG-modified IFN-λ, etc.

### Treating Acute Exacerbation of a Chronic Lung Disease

The present disclosure further provides methods of treating virus infection-induced acute exacerbation of a chronic lung disease, the methods generally involving administering to an individual in need thereof (e.g., an individual having a chronic lung disease) an effective amount of an EGF-R inhibitor. Suitable EGF-R inhibitors are as described above.

In some embodiments, the acute exacerbation of a chronic lung disease is caused by a respiratory virus, e.g., where the respiratory virus is a rhinovirus, an influenza virus, a respiratory syncytial virus, a parainfluenza virus, a metapneumovirus, a coronavirus, or an adenovirus.

Chronic lung diseases include, e.g., asthma, interstitial lung disease, idiopathic pulmonary fibrosis, chronic obstructive pulmonary disease (COPD; which includes chronic bronchitis and emphysema), cystic fibrosis, radiation-induced pulmonary fibrosis, sarcoidosis, pulmonary sarcoidosis, bronchiectasis, bronchiolitis, and the like. An individual having a chronic lung disease can experience an acute exacerbation of the disease if the individual has a viral infection. For example, a rhinovirus-16 infection can cause acute exacerbation of asthma.

In some embodiments, an effective amount of an EGF-R inhibitor is an amount that, when administered alone or in combination therapy, in one or more doses, is effective to reduce or ameliorate one or more symptoms associated with acute exacerbation, where such symptoms include, e.g., wheezing, coughing, dyspnea, chest tightness, etc.

In some embodiments, an effective amount of an EGF-R inhibitor is an amount that, when administered alone or in combination therapy, in one or more doses, is effective to increase pulmonary function in an individual experiencing virus infection-induced acute exacerbation of a chronic lung disease. For example, in some embodiments, an effective amount of an EGF-R inhibitor is an amount that, when given alone or in combination therapy, in one or more doses, increases one or more pulmonary functions by at least 10%, at least 15%, at least 20%, at least 25%, at least 50%, or more than 50%, compared to the pulmonary function in the absence of treatment with the EGF-R inhibitor.

Pulmonary function values are well known in the art. The following is an example of pulmonary function values that may be used. Other pulmonary function values, or combinations thereof, are intended to be within the scope of this invention. The values include, but are not limited to, FEV (forced expiratory volume), FVC (forced vital capacity), FEF (forced expiratory flow), Vmax (maximum flow), PEFR (peak expiratory flow rate), FRC (functional residual capacity), RV (residual volume), TLC (total lung capacity).

Suitable EGF-R inhibitors, dosages, formulations, and routes of administration; are described herein.

Where an EGF-R inhibitor is administered to an individual having a virus infection-induced exacerbation of a chronic lung disease, the EGF-R inhibitor can be administered alone (e.g., as monotherapy) or in combination therapy with one or more additional active agents used to treat the chronic lung disease. Suitable additional active agents include, e.g., an interferon (e.g., an interferon-alpha, an interferon-beta, an interferon-gamma, an interferon-lambda, an interferon-tau, an interferon-omega); a corticosteroid; a beta-2 agonist; an antihistamine; and the like).

For example, in the treatment of asthma, suitable active agents include: bronchodilators including beta-2-agonists including albuterol, levalbuterol, pirbuterol, artformoterol, formoterol, salmeterol, salbutamol, terbutaline, bitolterol, fluticasone, budesonide and anticholinergics including ipratropium, ipratropium bromide, oxitropium and tiotropium; corticosteroids, e.g., glucocorticoids (including oral, systemic and inhaled glucocorticoids), beclomethasone, budesonide flunisolide, fluticasone, mometasone, triamcinolone, methyprednisolone, prednisolone, prednisone, ciclesonide; leukotriene modifiers including montelukast, zafirlukast, pranlukast and zileuton; mast cell stabilizers including cromolyn, cromoglicate and nedocromil; epinephrine; ephedrine; methylxanthines including theophylline, aminophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; IgE blockers including Omalizumab; a tumor necrosis factor-alpha (TNF-α) inhibitor (e.g., Adalimuba; Certolizumab pegol; Etanercept; Golibumab; Infliximab; etc.); and combinations thereof.

As another example, in the treatment of interstitial lung disease, suitable additional active agents include: corticosteroid drugs; cytotoxic drugs such as azathioprine and cyclophosphamide; antioxidants such as acetylcysteine; and anti-fibrotics such as bosentan and pirfenidone.

As another example, in the treatment of COPD, suitable additional active agents include: beta-2-agonists such as albuterol and levalbuterol; anticholinergic bronchodilators such as ipratropium; combination drugs such as combivent, which contains albuterol and ipratropium; long-acting bronchodilators such as tiotropium, salmeterol, formoterol, and arformoterol; corticosteroids such as prednisone; antibiotics, and expectorants such as guaifenesin.

As another example, in the treatment of cystic fibrosis, suitable additional active agents include: bronchodilators such as albuterol, theophylline, ipratropium; mucolytics such as guaifenesin, DNase, hypertonic saline, and N-acetylcysteine; anti-inflammatives such as triamcinolone, flunisolide, fluticasone, beclomethasone, prednisone, methylprednisone, ibuprofen, montelukast, cromolyn; antibiotics such as ciprofloxacin, doxycycline, co-trimoxazole, tobramycin, cephalexin, colistin, ceftazidime, carbapenems (e.g., meropenem), piperacillin, dicloxacillin, and azithromycin.

As another example, in the treatment of sarcoidosis, suitable additional active agents include: glucocorticoids such as triamcinolone, prednisone, dexamethasone, triamcinolone and cortisone; monoclonal antibodies such as infliximab; immunosuppressive agents such as azathioprine; and amebicides such as chloroquine.

As another example, in the treatment of bronchiectasis, suitable additional active agents include: antibiotic such as azithromycin, amoxicillin, tobramycin, tetracycline, gentamicin, doxycycline, levofloxacin, amikacin, ceftazidime, carbapenems (e.g., meropenem), piperacillin, sulfamethoxazole-trimethoprim and tobramycin; bronchodilators such as albuterol; corticosteroid such as beclomethasone, fluticasone; mucolytics such as N-acetylcysteine; and expectorants such as guaifenesin.

### Formulations, Dosages, Routes of Administration

An active agent (also referred to herein as “drug”) is formulated with one or more pharmaceutically acceptable excipients. As noted above, “active agents” include, e.g., an EGF-R inhibitor, and in some embodiments, further include an additional therapeutic agent as described above. Where two or more active agents are administered, the two or more active agents can be formulated separately or can be co-formulated.

A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent may be administered to the host using any convenient means capable of resulting in the desired reduction in viral titers, symptoms of viral infection, etc. Thus, the active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of their pharmaceutically acceptable salts, or an active agent may be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles. An active agent is in some embodiments formulated into a preparation suitable for injection (e.g., subcutaneous, intravenous, intramuscular, intradermal, transdermal, or other injection routes) by dissolving, suspending or emulsifying the agent in an aqueous solvent (e.g., saline, and the like) or a nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent can be formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents. For enteral delivery, a subject formulation will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit, and shellac.

As one non-limiting example of a suitable oral formulation, an active agent can be formulated together with one or more pharmaceutical excipients and coated with an enteric coating, as described in U.S. Pat. No. 6,346,269. For example, a solution comprising a solvent, an active agent, and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for the active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™ copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

In some embodiments, an active agent is administered in an amount of from about 10 μg to about 500 mg per dose, e.g., from about 10 μg to about 20 μg, from about 20 μg to about 25 μg, from about 25 μg to about 50 μg, from about 50 μg to about 75 μg, from about 75 μg to about 100 μg, from about 100 μg to about 150 μg, from about 150 μg to about 200 μg, from about 200 μg to about 250 μg, from about 250 μg to about 300 μg, from about 300 μg to about 400 μg, from about 400 μg to about 500 μg, from about 500 μg to about 750 μg, from about 750 μg to about 1 mg, from about 1 mg to about 10 mg, from about 10 mg to about 25 mg, from about 25 mg to about 50 mg, from about 50 mg to about 100 mg, from about 100 mg to about 200 mg, from about 200 mg to about 300 mg, from about 300 mg to about 400 mg, or from about 400 mg to about 500 mg per dose.

In some embodiments, an active agent is administered in an amount of from about 10 mg/m² per dose to about 150 mg/m² per dose, e.g., from about 10 mg/m² per dose to about 15 mg/m² per dose, from about 15 mg/m² per dose to about 20 mg/m² per dose, from about 20 mg/m² per dose to about 25 mg/m² per dose, from about 25 mg/m² per dose to about 30 mg/m² per dose, from about 30 mg/m² per dose to about 35 mg/m² per dose, from about 35 mg/m² per dose to about 40 mg/m² per dose, from about 40 mg/m² per dose to about 50 mg/m² per dose, from about 50 mg/m² per dose to about 60 mg/m² per dose, from about 60 mg/m² per dose to about 70 mg/m² per dose, from about 70 mg/m² per dose to about 80 mg/m² per dose, from about 80 mg/m² per dose to about 90 mg/m² per dose, from about 90 mg/m² per dose to about 100 mg/m² per dose, from about 100 mg/m² per dose to about 110 mg/m² per dose, from about 110 mg/m² per dose to about 120 mg/m² per dose, from about 120 mg/m² per dose to about 130 mg/m² per dose, from about 130 mg/m² per dose to about 140 mg/m² per dose, or from about 140 mg/m² per dose to about 150 mg/m² per dose.

In some embodiments, an active agent is administered in an amount of from about 10 mg/m² per week to about 200 mg/m² per week, e.g., from about 10 mg/m² per week to about 15 mg/m² per week, from about 15 mg/m² per week to about 20 mg/m² per week, from about 20 mg/m² per week to about 25 mg/m² per week, from about 25 mg/m² per week to about 30 mg/m² per week, from about 30 mg/m² per week to about 35 mg/m² per week, from about 35 mg/m² per week to about 40 mg/m² per week, from about 40 mg/m² per week to about 50 mg/m² per week, from about 50 mg/m² per week to about 60 mg/m² per week, from about 60 mg/m² per week to about 70 mg/m² per week, from about 70 mg/m² per week to about 80 mg/m² per week, from about 80 mg/m² per week to about 90 mg/m² per week, from about 90 mg/m² per week to about 100 mg/m² per week, from about 100 mg/m² per week to about 110 mg/m² per week, from about 110 mg/m² per week to about 120 mg/m² per week, from about 120 mg/m² per week to about 130 mg/m² per week, from about 130 mg/m² per week to about 140 mg/m² per dose, from about 140 mg/m² per week to about 150 mg/m² per week, from about 150 mg/m² per week to about 160 mg/m² per week, from about 160 mg/m² per week to about 170 mg/m² per week, from about 170 mg/m² per week to about 180 mg/m² per week, from about 180 mg/m² per week to about 190 mg/m² per week, or from about 190 mg/m² per week to about 200 mg/m² per week.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, an active agent is administered for the lifetime of the individual.

In some embodiments, administration of an active agent is discontinuous, e.g., an active agent is administered for a first period of time and at a first dosing frequency; administration of the active agent is suspended for a period of time; then the active agent is administered for a second period of time for a second dosing frequency. The period of time during which administration of the active agent is suspended can vary depending on various factors, e.g., patient response; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

### Routes of Administration

An active agent is administered to an individual using any available method and route suitable for drug delivery, including systemic and localized routes of administration.

Conventional and pharmaceutically acceptable routes of administration include inhalational (e.g., intranasal), intramuscular, intratracheal, subcutaneous, intradermal, transdermal, topical (e.g. to the skin, to the eye, etc.), intravenous, rectal, oral, vaginal, ocular, intraocular, and other enteral and parenteral routes of administration. Routes of administration may be combined, if desired, or adjusted depending upon the agent and/or the desired effect. The compound can be administered in a single dose or in multiple doses.

The route of administration will in some embodiments depend on the nature of the viral infection. As an example, a respiratory viral infection (e.g., an infection with a virus that causes a respiratory disease) can be treated by administering an active agent to the respiratory tract, e.g., via inhalational route of administration, via intratracheal administration, via intranasal administration, etc. As another example, a viral infection with a virus that causes gastrointestinal disorder can be treated by administering an active agent to the GI tract, e.g., via oral administration, via rectal administration, or via intravenous administration. As another example, a viral infection that causes a skin disorder (e.g., warts, skin lesions, etc.) can be treated by administering an active agent to the skin via topical administration, or topically to a mucosal surface. As another example, a viral infection that infects vaginal tissues, genital tissues, the anus, etc., can be treated by administering an active agent directly to the affected tissue, e.g., intravaginal administration, rectal administration, perianal administration; oral administration to treat an oral mucosal infection, etc.

In some embodiments, direct application of the EGFR inhibitor to the mucosal surface that the virus is infecting is carried out. For example, in some embodiments, an EGFR inhibitor is administered via an inhaled route to treat a respiratory virus infection directly. As another example, for HPV or HIV, which are sexually transmitted viruses, an EGFR inhibitor is in some embodiments applied topically, e.g., to mucosal tissue. As another example, for viruses that affect the gastrointestinal system, oral administration is in some embodiments carried out, to provide the inhibitor to the target (gastrointestinal tract) epithelium. In some embodiments, e.g., where a viral incubation period is long (e.g., infection occurs before robust symptoms develop) and/or where a virus causes systemic illness, an oral agent is administered to treat the virus infection.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations. Inhalational routes of delivery are also contemplated, e.g., where the virus is one that infects the airways, lungs, etc.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection, and epidermal administration.

### Subjects Suitable for Treatment

Individuals in need of treatment with a subject treatment method include: a) individuals who have been exposed to a virus, but who have not yet been infected; b) individuals who have been infected with a virus, and who have not been treated with any anti-viral agent (e.g., infected and treatment naïve individuals); c) individuals who have been infected with a virus, who have been treated with an anti-viral agent other than an EGF-R inhibitor, and who have not responded to the anti-viral agent other than an EGF-R inhibitor; d) individuals who have been infected with a virus, who have been treated with an anti-viral agent other than an EGF-R inhibitor, and who have developed resistance to the anti-viral agent other than an EGF-R inhibitor; and e) individuals who have not yet been infected with a virus, but who are at risk of infection (e.g., due to possible or likely exposure to an infected individual; due to an immunocompromised status; and the like), e.g., individuals who are at greater risk than the general population of becoming infected; and individuals who are at greater risk, if infected, of developing complications or experiencing more severe symptoms, than the general population.

Individuals suitable for treatment with a subject method include, e.g., a human, where the human is from about one month to about 6 months, from about 6 months to about 1 year, or from about 1 year to about 5 years of age. Individuals suitable for treatment with a subject method include, e.g., a human, where the human is from about 5 years to about 12 years, from about 13 years to about 18 years, or from about 18 years to about 25 years of age. Individuals suitable for treatment with a subject method include, e.g., a human, where the human is from about 25 years to about 50 years, from about 50 years to about 75 years of age, or older than 75 years of age.

In some embodiments, an individual who is suitable for treatment with a subject treatment method is an individual who has not yet been infected with a virus, but who is at greater risk than the general population of becoming infected. Such individuals include, e.g., individuals who are possibly or likely exposed to a virus-infected individual, where such individuals include, e.g., medical personnel, military personnel, prison inmates, and any individual living in a population that includes at least one virus-infected individual.

Individuals who are at greater risk, if infected, of developing complications or experiencing more severe symptoms, than the general population, include, but are not limited to, immunocompromised individuals.

Individuals suitable for treatment with a subject method include, e.g., a human, where the human is immunocompromised. Immunocompromised individuals include, e.g., individuals infected with a human immunodeficiency virus, e.g., where the individual has a lower than normal CD4⁺ T cell count. The normal range of CD4⁺ T cell for humans is from about 600 to about 1500 CD4⁺ T lymphocytes per mm³ blood. Thus, in some embodiments, an immunocompromised individual has a CD4⁺ T cell count that is less than about 600 CD4⁺ T cells per mm³ blood.

Immunocompromised individuals include individuals who are immunocompromised as a result of treatment with a cancer chemotherapeutic agent; and individuals who are immunocompromised as a result of radiation therapy (e.g., for the treatment of a cancer). Immunocompromised individuals include individuals who are immunocompromised due to chronic disease, e.g., cancer, diabetes mellitus, rheumatologic diseases (e.g., systemic lupus erythematosus, etc.), immunoglobulin deficiency diseases, and the like. Immunocompromised individuals include transplant recipients (e.g., lung transplant recipients, kidney transplant recipients, bone marrow transplant recipients, etc.). Immunocompromised individuals include individuals who are immunocompromised as a result of taking certain medications such as steroids, chemotherapeutic agents, TNF-α inhibitors, and the like.

Individuals suitable for treatment with a subject method include individuals who are immunosuppressed, e.g., individuals who are undergoing immunosuppressive treatment, where such individuals include, e.g., transplant recipients. Transplant recipients include, e.g., allograft recipients, and the like. Immunosuppressive treatments include, e.g., treatment with FK506.

Individuals suitable for treatment with a subject method include virus-infected individuals who have a chronic lung disease, e.g., asthma, COPD, cystic fibrosis, emphysema, chronic bronchitis, interstitial lung disease, bronchitis; sarcoidosis, idiopathic pulmonary fibrosis, bronchiectasis, acute respiratory distress syndrome (ARDS), and acute lung injury. Individuals suitable for treatment with a subject method include virus-infected individuals who have received a lung transplant. Individuals suitable for treatment with a subject method include individuals who have a chronic disease that has lung involvement, where such diseases include, e.g., systemic lupus erythematosus, Sjögren's disease, rheumatoid arthritis, and connective tissue diseases.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); PFU, plaque-forming units; TCID50, tissue culture-infective dose 50%; and the like.

### Example 1

### Effect of EGF-R Inhibitor on Rhinovirus

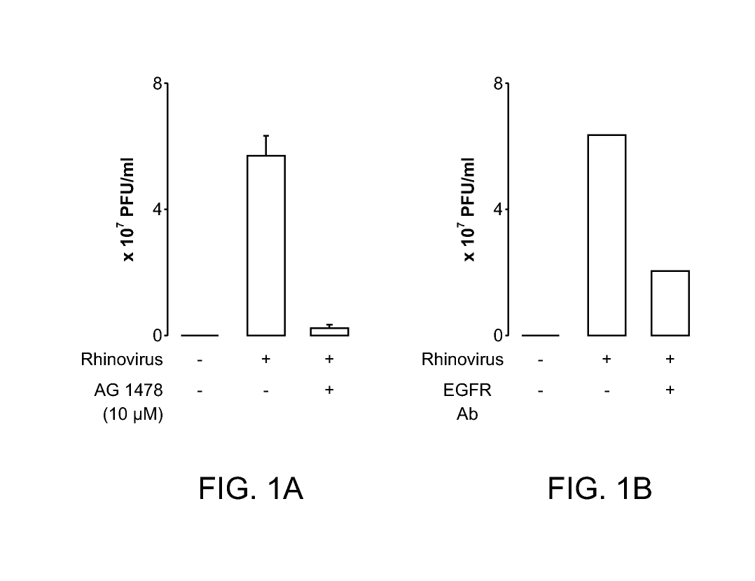

The effect of an EGF-R inhibitor on rhinovirus infectivity was assayed. First, human airway epithelial (NCI-H292 American Type Culture Collection Catalog No. CRL-1848) cells were treated with Rhinovirus 16 (major group Rhinovirus (RV) at multiplicity of infection (MOI) 10) or RV16 plus a selective EGFR tyrosine kinase inhibitor (AG 1478) at 10 μM. Cell lysates from these NCI-H292 cell cultures were collected at 6 hours post-infection. To quantitate Rhinovirus infection in airway epithelial cells, cell lysates were analyzed by viral plaque assay, a technique that is commonly utilized to measure viral infection. Next, serial dilutions of NCI-H292 cell lysates were placed on HeLa cell cultures for a standard plaque assay. Cell cultures were covered with an agarose overlay. Cultures were incubated for three days. After three days, cell cultures were stained with crystal violet and the number of plaques was counted. The results are shown in FIG. 1A. The results demonstrate that an EGF-R inhibitor can suppress rhinovirus infection. FIG. 1A. Serum-free medium alone (control, first column) 0 PFU/ml, Rhinovirus alone (second column) 5.7 plaque-forming units (PFU) per milliliter (PFU/ml), and Rhinovirus plus the selective EGFR inhibitor AG 1478 (10 μM, third column); n=2. In addition, the addition of a neutralizing antibody to EGFR decreased Rhinovirus infection.

The effect of an antibody specific for EGF-R on Rhinovirus 16 (RV16) infection was also assessed. Using a plaque assay as described above, NCI-H92 cells were treated with RV16 alone, or together with a neutralizing antibody specific for EGFR (“EGFR Ab”). The results are shown in FIG. 1B. FIG. 1B. Serum-free medium alone (control, first column) 0 PFU/ml, Rhinovirus alone (second column) 6.33 PFU/ml, and Rhinovirus plus anti-EGFR Ab (4 μg/ml, third column); n=1.

FIGS. 1A and 1B. Viral quantification of Rhinovirus infection by plaque assay. NCI-H292 cells infected with serum-free medium alone (control) or Rhinovirus (MOI=10) for 24 h with (A) the selective EGFR inhibitor AG 1478 (10 μM) and (B) EGFR neutralizing Ab (4 μg/ml). Cell lysates were collected after 24 h and Rhinovirus infection was quantified by plaque assay [(A) n=2±SEM; (B) n=1].

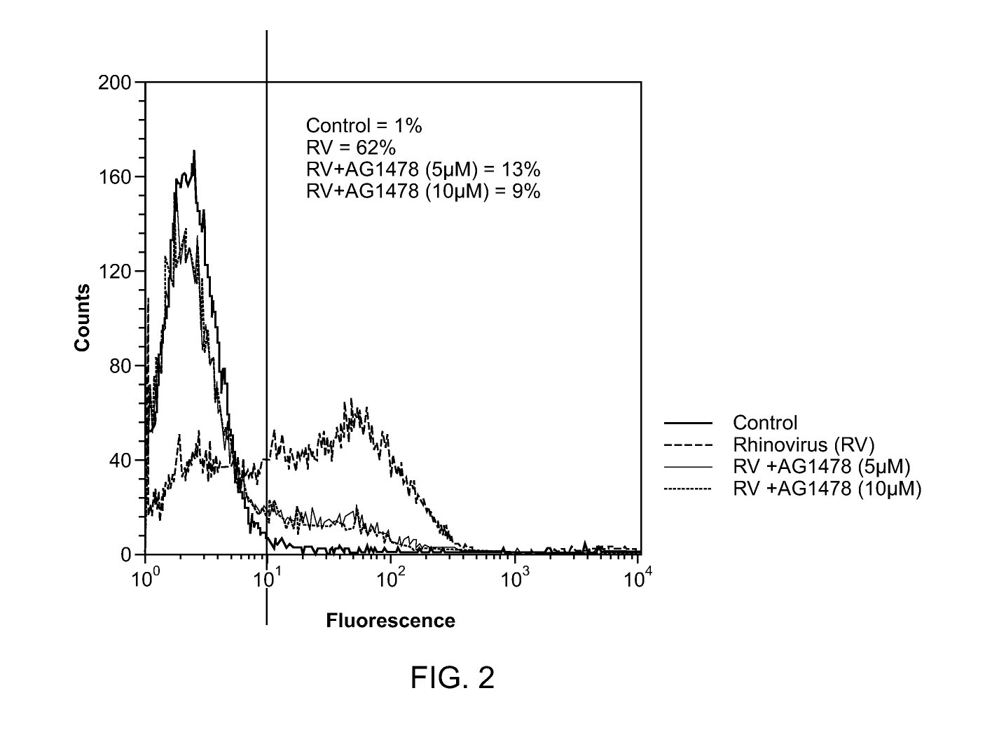

HeLa is an epithelial cell line that is routinely used to evaluate Rhinovirus infection. HeLa; American Type Culture Collections (ATCC) No. CCL-2. Here, HeLa cell cultures were treated with serum-free medium (Control), or with serum-free medium containing Rhinovirus 16 (major group RV at multiplicity of infection (MOI) 10) with and without a selective EGFR tyrosine kinase inhibitor (AG 1478). After 6 h, cell cultures were collected and stained with a monoclonal antibody that selectively binds to Rhinovirus. Cells were processed for flow cytometry and Rhinovirus infection in HeLa cells was measured. The data are representative of three separate experiments and are shown in FIG. 2.

The data, shown in FIG. 2, are expressed as percent infected cells. Control (no RV): 1%; RV: 62%; RV+AG1478 (5 μM): 13%; and RV+AG1478 (10 μM): 9%. The data show that EGF-R inhibitor significantly decreases rhinovirus infection in epithelial cells. No effect with a platelet derived growth factor (PDGF) inhibitor (another tyrosine kinase inhibitor) was observed.

### Example 2

### EGFR Inhibition Suppresses Influenza Virus Infection

Human airway epithelial (NCI-H292; American Type Culture Collection Catalog No. CRL-1848) cell line infected with Influenza A/H1N1/PR8 (MOI˜1) were treated with or without a selective EGFR inhibitor, AG1478 (10 μM), for 24 h on 8-well slides. Cell cultures were fixed and stained after 24 h utilizing an Influenza A-specific monoclonal antibody (MAb) (sc-52025, 1:50 dilution; Santa Cruz Biotechnology). First, images were obtained at 40× magnification. Next, images were obtained at 20× magnification in eight randomly selected fields and the number of cells stained was counted in four independent experiments. The control condition (serum-free medium alone) showed no staining for infected cells.

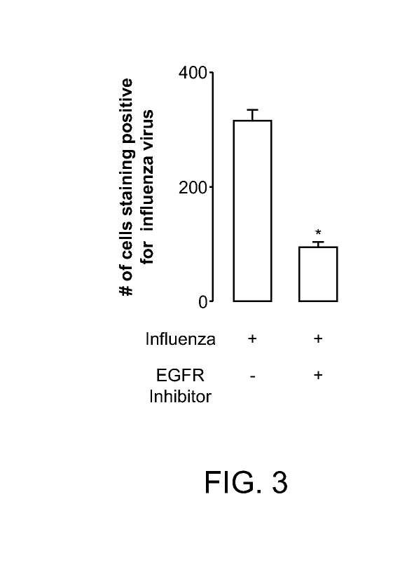

The results are shown in FIG. 3. It was found that the human airway epithelial (NCI-H292) cell line treated with a selective EGFR tyrosine kinase inhibitor, AG 1478 (10 μM), significantly decreased Influenza A/H1N1/PR8 infection at 24 h. It was found that NCI-H292 cells treated with AG 1478 (10 μM) decreased Influenza A/PR8 infection significantly [Influenza alone, 316±19 cells vs Influenza+AG 1478, (10 μM) 94±49 cells; n=4; p<0.0001] at 24 h.

FIG. 3. Airway epithelial cells NCI-H292 cells infected with Influenza A/PR8 (MOI˜1) alone or with the addition of the selective EGFR tyrosine kinase inhibitor AG1478 (10 μM). Cells were fixed and stained at 24 h and cells staining positive for Influenza were counted [Influenza A monoclonal Ab (sc-52025), 1:50 dilution; Santa Cruz Biotechnology; n=4; p<0.0001].

### Example 3

### EGFR Inhibition Suppresses Respiratory Syncytial Virus (RSV) Infection

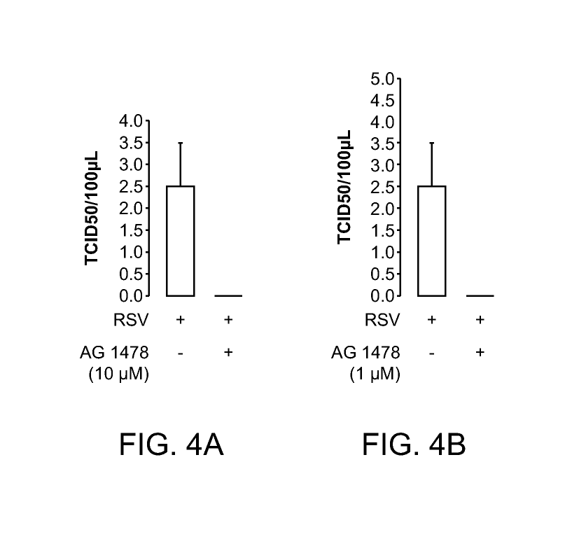

To study the effect of a selective EGFR inhibitor on RSV infection, viral quantification by tissue culture-infective dose (TCID50)/100 μL in epithelial HeLa cell cultures treated with or without the selective EGFR tyrosine kinase inhibitor AG 1478 was used. The data are shown in FIGS. 4A and 4B, and show that AG 1478 (1 μM and 10 μM) suppressed RSV virus infection significantly. FIG. 4A, RSV alone 10^{2.5±1} TCID50 vs RSV plus AG 1478 (10 μM) 0 TCID50; n=2. FIG. 4B, RSV alone 10^{4.38±0.1} TCID50 vs RSV plus AG 1478 (1 μM) 0 TCID50; n=2.

FIGS. 4A and 4B. Viral quantification by tissue culture-infective dose (TCID50)/100 μL in epithelial cell cultures treated with or without the selective EGFR inhibitor, AG 1478 [(A) AG1478 (10 μM) in TCID50/100 μL at 2 days ±SEM, (n=2 separate experiments); (B) AG 1478 (1 μM) in TCID50/100 μL at 4 days ±SEM, (n=2 separate experiments).

A similar set of experiments was conducted, to test the effect of Gefitinib (Iressa; ZD 1839; N-(3-chloro-4-fluoro-phenyl)-7-methoxy-6-(3-morpholin-4-ylpropoxy)quinazolin-4-amine) on RSV infection. Gefitinib (1 and 10 μM) suppressed RSV virus infection. The data are shown in FIG. 5 (RSV alone 10^{1.5} TCID50 (first column), RSV plus Gefitinib (1 μM) 0 TCID50 (second column), and RSV plus Gefitinib (10 μM) 0 TCID50 (third column); n=1).

FIG. 5. Viral quantification by tissue culture-infective dose (TCID50)/100 μL at 2 days in epithelial cell cultures treated with or without the selective EGFR inhibitor, Gefitinib. RSV alone (first column), RSV plus Gefitinib (1 μM, second column), and RSV plus Gefitinib (10 μM, third column); n=1.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a viral infection in an individual, the method comprising administering to the individual an effective amount of an epidermal growth factor receptor (EGF-R) antagonist, wherein the viral infection is caused by a virus of the family Picornaviridae, Orthomyxoviridae, Paramyxoviridae, Coronaviridae, Adenoviridae, Reoviridae, Caliciviridae, Astroviridae, Retroviridae, Herpesviridae, or Papillomaviridae.

2. The method of claim 1, wherein the individual is a human.

3. The method of claim 1, wherein the individual is immunocompromised.

4. The method of claim 1, wherein the individual has a chronic lung disease.

5. The method of claim 4, wherein the chronic lung disease is asthma, chronic obstructive pulmonary disease, cystic fibrosis, interstitial lung disease, bronchitis, sarcoidosis, idiopathic pulmonary fibrosis, bronchiectasis, acute respiratory distress syndrome, or acute lung injury.

6. The method of claim 1, wherein the individual is from about 1 month to about 6 months of age, or from about 6 months to about 1 year of age.

7. The method of claim 1, wherein the individual is from about 1 year to about 5 years of age, or from about 5 years to about 12 years of age.

8. The method of claim 1, wherein the individual is from about 13 years to about 19 years of age.

9. The method of claim 1, wherein the individual is from about 20 years to about 30 years of age.

10. The method of claim 1, wherein the individual is from about 30 years to about 50 years of age.

11. The method of claim 1, wherein the individual is from about 50 years to about 75 years of age.

12. The method of claim 1, wherein the virus infection is caused by a coxsackievirus, a rhinovirus, an enterovirus, or hepatitis virus A.

13. The method of claim 1, wherein the virus infection is caused by an influenza virus.

14. The method of claim 1, wherein the virus infection is caused by a paramyxovirus, a pneumovirus, a metapneumovirus, a mobillivirus, a rubulavirus, or a respiratory syncytial virus.

15. The method of claim 1, wherein the virus infection is caused by HCoV-229E, HcoV-OC43 (HCoV-OC43), or SARS-CoV.

16. The method of claim 1, wherein the virus infection is caused by a human adenovirus.

17. The method of claim 1, wherein the virus infection is caused by a rotavirus.

18. The method of claim 1, wherein the virus infection is caused by Norwalk virus.

19. The method of claim 1, wherein the virus infection is caused by an astrovirus.

20. The method of claim 1, wherein the virus infection is caused by a lentivirus.

21. The method of claim 1, wherein the virus infection is caused by herpes simplex virus-1, herpes simplex virus-2, varicella zoster virus, human herpes virus-6, Epstein-Barr virus, or human herpes virus-8.

22. The method of claim 1, wherein the virus infection is caused by a human papilloma virus.

23. The method of claim 1, wherein the EGF-R antagonist is a small molecule inhibitor.

24. The method of claim 23, wherein the EGF-R antagonist selectively inhibits EGF-R tyrosine kinase activity.

25. The method of claim 23, wherein the EGF-R antagonist is a multi-kinase inhibitor.

26. The method of claim 23, wherein the EGF-R antagonist is a quinazoline or quinazoline derivative.

27. The method of claim 23, wherein the EGF-R antagonist is a diaminophthalimide.

28. The method of claim 23, wherein the EGF-R antagonist is a tyrphostin.

29. The method of claim 23, wherein the EGF-R antagonist is a pyrrolopyrimidine.

30. The method of claim 23, wherein the EGF-R antagonist is a bicyclic heterocyclic compound.

31. The method of claim 1, wherein the EGF-R antagonist is an antibody specific for EGF-R.

32. The method of claim 1, wherein the EGF-R antagonist is an inhibitory nucleic acid that reduces the level of EGF-R produced in a cell.

33. The method of claim 1, wherein the EGF-R antagonist is administered via a systemic route.

34. The method of claim 1, wherein the EGF-R antagonist is administered intravenously.

35. The method of claim 1, wherein the EGF-R antagonist is administered to the respiratory system of the individual.

36. The method of claim 35, wherein the EGF-R antagonist is administered via inhalation.

37. The method of claim 35, wherein the EGF-R antagonist is administered intranasally.

38. The method of claim 1, wherein the EGF-R antagonist is administered topically.

39. The method of claim 38, wherein the EGF-R antagonist is administered to the skin.

40. The method of claim 38, wherein the EGF-R antagonist is administered to the eye.

41. The method of claim 1, wherein the EGF-R antagonist is administered vaginally, rectally, or perianally.

42. The method of claim 1, wherein the EGF-R antagonist is administered orally.

43. The method of claim 1, wherein the EGF-R antagonist is administered after the individual has been exposed to the virus.

44. The method of claim 1, wherein said administration is effective to reduce viral load in the individual.

45. The method of claim 1, wherein said administration is effective to ameliorate at least one symptom of viral infection in the individual.

46. The method of claim 45, wherein said at least one symptom is fever, diarrhea, cough, shortness of breath, or vomiting.

47. The method of claim 1, further comprising administering at least one additional therapeutic agent to the individual.

48. A method of treating respiratory virus-induced exacerbation of a chronic lung disease in an individual, the method comprising administering to the individual an effective amount of an epidermal growth factor receptor (EGF-R) antagonist.

49. The method of claim 48, wherein the respiratory virus is a rhinovirus, an influenza virus, a respiratory syncytial virus, a parainfluenza virus, a metapneumovirus, a coronavirus, or an adenovirus.

50. The method of claim 48, wherein the chronic lung disease is asthma, chronic obstructive pulmonary disease, cystic fibrosis, interstitial lung disease, bronchitis, sarcoidosis, idiopathic pulmonary fibrosis, bronchiectasis, or bronchiolitis.

51. The method of claim 48, further comprising administering at least one additional therapeutic agent to the individual.

52. The method of claim 51, wherein the at least one additional therapeutic agent is an interferon.

53. The method of claim 52, wherein the interferon is an interferon-alpha, an interferon-beta, an interferon-gamma, an interferon-lambda, an interferon-tau, or an interferon-omega.
